(19) United States
(12) Patent Application Publication
(10) Pub. No.: US 2026/0218224 A1
(43) Pub. Date: Jul. 30, 2026

(54) MANIPULATING AUTOPHAGY TO ENHANCE NUTRIENT UTILIZATION AND CROP RESILIENCE

(71) Applicant: Wisconsin Alumni Research Foundation, Madison, WI (US)

(72) Inventors: Marisa Otegui, Madison, WI (US); Sushmita Roy, Madison, WI (US); Maria Charlotte Steelheart Molina, Madison, WI (US)

(21) Appl. No.: 19/459,963

(22) Filed: Jan. 26, 2026

Related U.S. Application Data
(60) Provisional application No. 63/749,220, filed on Jan. 24, 2025.

(51) Int. Cl. C12N 15/82; A01N 63/60; A01P 21/00; C07K 14/415

(52) U.S. Cl. CPC C12N 15/8261; A01N 63/60; A01P 21/00; C07K 14/415; C12N 15/8271

(57) ABSTRACT

The present invention provides constructs for enhancing the autophagy pathway in plants. The constructs comprise polynucleotides encoding the proteins Autophagy-related protein 1 (ATG1), Autophagy-related protein 9 (ATG9), Autophagy-related protein 12 (ATG12), Autophagy-related protein (ATG8a), and bZIP transcription factor 87 (bZIP87). Plants comprising the constructs and methods of using the plants are also provided.

### CROSS-REFERENCE TO RELATED APPLICATIONS

This patent application claims the benefit of U.S. Provisional Application No. 63/749,220 filed on Jan. 24, 2025, the entire contents of which are incorporated herein by reference.

### STATEMENT REGARDING FEDERALLY SPONSORED RESEARCH

This invention was made with government support under 1840687 awarded by the National Science Foundation. The government has certain rights in the invention.

### SEQUENCE LISTING

The contents of the electronic sequence listing (96029604740_SL.xml; Size: 17,084 bytes; and Date of Creation: Jan. 19, 2026) are herein incorporated by reference in their entirety.

### BACKGROUND

Current agricultural practices heavily rely on the application of fertilizers, which are both expensive and polluting, contributing to 5% of the global greenhouse gas emissions. Plants can recycle cellular components to maximize growth under low nutrient conditions through a cellular process called autophagy. Autophagy is the primary pathway that controls the degradation of organelles and cytoplasmic components for nutrient recycling and cellular protection in all eukaryotes. In animals, the targeted activation of autophagy through intermittent fasting, exercise, and dietary modifications results in multiple health benefits and prolonged lifespan. Whereas autophagy is an essential process for animal survival, it is not for plants because plants have developed additional multilayered cellular recycling pathways that allow them to adjust their growth to environmental cues and survive under fluctuating environments. Most of these adaptive strategies reduce growth under unfavorable conditions.

Accordingly, there is a remaining need in the art for a more detailed analysis of autophagy components and their regulation in plants to establish methods for recycling nutrients more efficiently, increasing plant productivity, improving stress resistance, and decreasing fertilizer application.

### SUMMARY

Genetically modified plant cells and methods of using and generating these plant cells and plants derived from the cells are provided. The methods and plants produced by the methods demonstrate increased stress tolerance and yield for the plants and decreased fertilizer requirements for the plants. The genetically modified plant cells and plants comprise a heterologous promoter operably connected to a polynucleotide encoding at least one protein selected from the group consisting of ATG1, ATG8 (ATG8a), ATG9, ATG12, bZIP87, and combinations thereof.

In another aspect, methods of increasing at least one phenotypic trait of a genetically modified plant as compared to the non-genetically modified control plant are provided. The genetically modified plant is modified to over-express at least one autophagy protein including at least one of ATG1, ATG8, ATG9, ATG12, bZIP87. The methods may include introducing an expression cassette with a promoter operably connected to a polynucleotide encoding at least one autophagy protein including at least one of ATG1, ATG8, ATG9, ATG12, bZIP87 into cells of the plant and growing the plant. The phenotypic trait increased may be one of biomass, yield, stress tolerance, heat tolerance, and low fertilizer tolerance or a decrease in need for fertilizer or amount of fertilizer.

In another aspect, methods of decreasing the amount of fertilizer used to grow plants are provided. These methods include growing a genetically modified plant that overexpresses at least one of ATG1, ATG8, ATG9, ATG12, bZIP87 as compared to a control non-modified plant. The plant may comprise a promoter operably connected to a polynucleotide encoding at least one of ATG1, ATG8, ATG9, ATG12, and bZIP87. The genetically modified plant may be provided at least 10% less fertilizer as compared to a control plant to produce similar results.

In yet another aspect, methods of generating plants with increased stress tolerance or yield are provided. The methods include growing a genetically modified plant that overexpresses at least one of ATG1, ATG8, ATG9, ATG12, bZIP87 as compared to a control non-modified plant. The plant may comprise a promoter operably connected to a polynucleotide encoding at least one of ATG1, ATG8, ATG9, ATG12, and bZIP87. The genetically modified plant may have increased stress tolerance or increased yield as compared to a control plant.

### BRIEF DESCRIPTION OF THE DRAWINGS

FIG. 1 illustrates the higher biomass phenotype of 4-week old W22 maize seedlings overexpressing YFP-Atg8a under either high (15 mM NO₃⁻) or low N (0.15 mM NO₃⁻), as compared to the corresponding wild-type W22 seedlings under the same N treatment.

FIG. 2 shows higher biomass production in the 4-week-old plants of FIG. 1. Plants overexpressing YFP-Atg8a exhibit higher biomass than the wild-type W22 control plants, under either (A) high Nitrogen (15 mM NO₃⁻) or (B) low Nitrogen (0.15 mM NO₃⁻). Biomass was measured by calculating shoot fresh weight (gr). Asterisks denote statistical differences between WT (W22) and YFP-Atg8 seedling (t-test; p<0.05). Bars indicate standard deviation.

FIG. 3 illustrates the higher chlorophyll phenotype of 4-week old maize seedling leaves overexpressing YFP-Atg8a. Higher chlorophyll can be observed under both high N (15 mM NO₃⁻) (bottom, left) or low N (0.15 mM NO₃⁻) (bottom, right) treatments, as compared to wild-type W22 seedlings for each N treatment (top).

FIG. 4 shows higher total chlorophyll content of the 4-week-old plants of FIG. 3. Higher chlorophyll content (mg/g fresh weight (FW)) was observed under both (A) high N (15 mM NO₃⁻) and (B) low N (0.15 mM NO₃⁻) treatments, as compared to wild-type W22 seedlings for each N treatment. Asterisk denotes statistical differences between WT (W22) and YFP-Atg8 seedling (t-test; p<0.05). Bars indicate standard deviation.

FIG. 5 illustrates field-grown maize plants overexpressing mCherry-Atg9 (top, right) or YFP-Atg8a (bottom, right), as compared to control plants expressing only mCherry (top, left) or wild-type W22 plants (bottom, left), respectively. This experiment was conducted under conventional growing conditions (high N).

FIG. 6 shows biomass accumulation of the plants of FIG. 5. Biomass measurements were collected on (A) shoot height (cm) per plant, (B) shoot dry weight (gr) (DW=stem DW and total leaf DW), and (C) total chlorophyll content (mg/g FW). Asterisk denotes statistical differences between both overexpressing mCherry-Atg9 and YFP-Atg8a, as compared to their controls, mCherry and wild-type W22, respectively (t-test; p<0.05). Plants expressing mCherry-Atg9 exhibited significantly greater plant height and shoot dry weight than mCherry control plants. Plants expressing YFP-Atg8a exhibited significantly greater plant height but lower shoot dry weight than W22 control plants. No significant differences were observed for total chlorophyll for either mCherry-Atg9 or YFP-Atg8a plants, as compared to their respective control plants. Bars indicate standard deviation.

FIG. 7 shows several phenotypes of maize plants overexpressing YFP-Atg8a and mCherry-Atg9 as grown in the field. Phenotypes include (A) pollen production (cm³) per plant, (B) number of days for tasseling per plant, and (C) the period between the release of pollen and the formation of stigmas (ASI) per plant. Plants expressing mCherry-Atg9 or YFP-Atg8a produced a significantly greater volume of pollen, a smaller number of days to tasseling, and a smaller ASI, as compared to mCherry and W22 control plants, respectively. Asterisk denotes statistical differences between both overexpressing mCherry-Atg9 and YFP-Atg8a and their control mCherry and W22 (t-test; p<0.05).

FIG. 8 illustrates more fully developed ears harvested from field-grown maize plants overexpressing mCherry-Atg9 (top, right) or YFP-Atg8a (bottom, right), as compared to mCherry or W22 control plants (left), respectively, under high N.

FIG. 9 shows grain yield components as measured from the ears of FIG. 8. Yield components include (A) number of ears per plant, (B) number of kernels (grains) per plant, and (C) dry weight of 20 kernels (gr) per plant. Plants expressing mCherry-Atg9 or YFP-Atg8a produced a significantly greater number of ears per plant, a greater number of kernels per plant, and a greater 20-kernel dry weight per plant, as compared to mCherry and W22 control plants, respectively. Asterisk denotes statistical differences between both overexpressing mCherry-Atg9 and YFP-Atg8a and their control mCherry and W22 (t-test; p<0.05).

FIG. 10 shows quantification of phenotypic parameters of maize plants overexpressing mCherry-Atg9 (mCh-Atg9) or mCherry alone (mCh) grown in the field. Three independent transformation events were analyzed. Biomass accumulation measured as plant height (A), fresh weight (FW) and water content of stem (B and C), leaves per plant (D), and FW and water content of leaves (E and F). Asterisk denotes statistical differences between overexpressing mCherry-Atg9 and mCherry lines (n=10, t-test; p<0.05). Bars indicate standard deviation.

FIG. 11 shows yield of maize plants overexpressing mCherry-Atg9 or mCherry and grown in the field. The yield was measured as the number of ears (A and B), ear fresh weight (FW) (C), number of kernels (grains) per plant (D), dry weight (DW) of 100 kernels (gr) per plant (E) and total yield (Kernel DW) per plant (F). Asterisk denotes statistical differences between mCherry-Atg9 and mCherry control plants (n=10, t-test; p<0.05).

FIG. 12 shows phenotypic characterization of maize plants overexpressing YFP-Atg8a grown in the field. Only one transformation event is available for YFP-Atg8a. Biomass accumulation measured as plant height (A), fresh weight (FW) and water content of stem (B and C), leaves per plant (D), and FW and water content of leaves (E and F). Asterisk denotes statistical differences between both overexpressing YFP-Atg8 and W22 (n=10, t-test; p<0.05). Bars indicate standard deviation.

FIG. 13 shows yield of maize plants overexpressing YFP-Atg8a in the field. The yield was measured as the number of ears (A and B), ear FW (C), number of kernels (grains) per plant (D), and the dry weight (DW) of 100 kernels (gr) per plant (E) and total yield per plant (F). Asterisk denotes statistical differences between overexpressing YFP-Atg8a control W22 (n=10, t-test; p<0.05).

FIG. 14 shows the phenotype of maize plants overexpressing mCherry-Atg9 grown under nitrogen starvation. Biomass accumulation in control condition for mCherry and mCherry-Atg9 (A and B, respectively), and under nitrogen starvation for mCherry and mCherry-Atg9 (C and D, respectively), for three independent transformation events measured as plant height (E), leaf area (F), chlorophyll content as SPAD values (G) and leaf area over time (H). Asterisk denotes statistical differences between plants overexpressing mCherry-Atg9 and mCherry (n=5, t-test; p<0.05). Bars indicate standard deviation.

FIG. 15 shows the phenotype of maize plants overexpressing YFP-Atg8a grown under nitrogen starvation. Biomass accumulation in control conditions for W22 and YFP-Atg8a (A and B, respectively), and under nitrogen starvation for W22 and YFP-Atg8a (C and D, respectively), measured as plant height (E), leaf area (F), chlorophyll content as SPAD values (G) and leaf area over time (H). Asterisk denotes statistical differences between both overexpressing mCherry-Atg9 and mCherry (n=5, t-test; p<0.05). Bars indicate standard deviation.

FIG. 16 shows pollen and ear production of maize plants overexpressing mCherry-Atg9 or YFP-Atg8a, grown under nitrogen starvation. Pollen volume (A), ears with kernels (seeds) per plant (B), and number of kernels per plant (C and B) for mCherry and mCherry-Atg9 lines under either control or nitrogen starvation conditions. Pollen volume (E) and number of ears per plant (F) for W22 and YFP-Atg8a under control and nitrogen starvation conditions. The asterisks represent significant differences between genotypes for the control and nitrogen starvation treatments (n=5, t-test; p<0.05).

FIG. 17 shows the phenotypes of maize seedlings overexpressing Zip87, Atg1a, and Atg12 grown under either high (15 mM NO₃⁻) or low N (0.15 mM NO₃⁻). Images of 2^{nd} leaves (A), and graphs showing chlorophyll content (B), shoot height (C), and shoot FW (D) in 4-weeks-old plants under control or N starvation conditions. Asterisk denotes statistical differences between control line (mCherry E7) and the overexpressing lines seedling (n=4, t-test; p<0.05). Bars indicate standard deviation.

### DETAILED DESCRIPTION

The present invention provides novel DNA constructs for producing transgenic plants. The DNA constructs encode autophagy proteins and transcriptional regulators involved in autophagy in plants. Plants and plant cells comprising the constructs and methods of using the genetically modified plants are also provided.

In the present application, the inventors have developed novel constructs and methods for increasing autophagy in plants. By conducting multi-omic and gene network analyses, the inventors identified key genes that mediate various steps in autophagy, including autophagy genes and transcription factors of autophagy, in plants. The proteins encoded by these key genes include Autophagy-related Protein 1 (ATG1), Autophagy-related Protein 8a (ATG8a), Autophagy-related Protein (ATG9), Autophagy-related Protein 12 (ATG12), and bZIP transcription factor 87 (bZIP87). As described in the Examples, the inventors developed constructs comprising a heterologous promoter and one or more of the autophagy genes, transcriptional regulators of autophagy, or combinations thereof. The constructs were then expressed in trans in plant cells and plants to increase protein expression, and the phenotypes of the resulting plants were measured. More specifically in the initial examples, at least one of ATG8a and ATG9 genes were fused to marker genes (YFP or mCherry) and expression was driven by the heterologous maize Ubiquitin-1 promoter (ZmUbi1) to allow tracking of expression and increase their expression, respectively, as compared to the wild-type control plants. The constructs were transformed into maize and backcrossed to inbred lines W22 and LH244. Further constructs for expressing each of the listed proteins without a corresponding marker fusion were generated and plants generated from these constructs were also evaluated and found to perform similarly.

The transgenic plants were then grown in the greenhouse and under field conditions and subjected to variable rate fertilizer application treatments. Phenotypic traits of commercial importance (e.g., biomass, yield, stress resistance, and low fertilizer tolerance) were measured, as compared to the control plants. The inventors discovered that transformed greenhouse-grown plants exhibited greater biomass, yield and stress resistance than the control plants under low N. The inventors are also conducting similar experiments under low N in the field. These results indicate that overexpressing autophagy proteins leads to an increase in nutrient utilization efficiency, which will allow for a reduction in fertilizer application while still achieving the same phenotype or even greater productivity as compared to non-transformed plants as shown in the Examples. Thus, the constructs and methods of the invention provide additional genetic tools for increasing autophagy and reducing fertilizer application in maize. Several of these genes and proteins are highly conserved in sorghum (over 88% identity), and rice and homologs of the proteins described here may be overexpressed in these plants as well.

### Modified Plants and Plant Cells

The present invention provides a genetically modified plant cell comprising a promoter operably connected to a polynucleotide encoding at least one protein selected from the group consisting of ATG1, ATG8a, ATG9, ATG12, bZIP87, and combinations thereof. The promoter may be a heterologous promoter to allow for over-expression of the proteins.

As demonstrated in the Examples, generating plant cells and plants genetically modified to increase the expression of several autophagy proteins by expressing them in trans using the maize ZmUbi1 heterologous promoter resulted in plants having desirable phenotypic traits such as a reduced need for fertilizer. The key proteins identified mediate various steps in autophagy in plants, including autophagy proteins Autophagy-related Protein 1 (ATG1), Autophagy-related Protein 9 (ATG9), Autophagy-related Protein 12 (ATG12), Autophagy-related Protein 8a (ATG8a), and bZIP transcription factor 87 (bZIP87). These proteins were overexpressed in plant cells by introducing the polynucleotides encoding these proteins into plant cells. The polynucleotides were operably connected to a heterologous promoter to allow for increased expression as compared to a plant or plant cells to which the polynucleotides encoding at least one protein selected from ATG1, ATG8a, ATG9, ATG12, bZIP87, or combinations thereof, were introduced. In the Examples, the inventors utilized proteins from maize to genetically modify maize plants. However, the invention may be used with a “homolog” protein, a protein related to a second protein by descent from a common ancestral protein. In addition, while the inventors used maize proteins in their constructs, “ortholog” proteins, or proteins separated by speciation, may also be used. For example, maize genes may be used in a distinct crop plant such as sorghum, or sorghum autophagy protein orthologs may be used in maize.

As used herein, the terms “protein” or “polypeptide” or “peptide” may be used interchangeably to refer to a polymer of amino acids. A “protein” as contemplated herein typically comprises a polymer of naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine).

In some embodiments, one or more of these specific proteins, or a variant(s) thereof, is/are utilized. As used herein, a “variant” refers to a protein having an amino acid sequence that differs from a disclosed protein reference. A variant may have one or more insertions, deletions, or substitutions of an amino acid residue relative to a reference molecule. For example, a disclosed protein variant may have one or more insertion, deletion, or substitution of at least one amino acid residue relative to the reference disclosed proteins (SEQ ID NOs: 2, 4, 6, 8, and 10) disclosed herein. A variant may also refer to a polynucleotide having a sequence that differs from a disclosed polynucleotide reference. One or more variant(s) may arise due to natural genetic variation between lines or cultivars of the same species or due to artificial sequence variation due to sequencing errors or instrument biases. Such variation manifests in sequence databases, such as NCBI, as multiple accession versions of the same gene or protein. It is important to account for such unavoidable variation that is real or artificial.

A “deletion” in a disclosed protein or polynucleotide refers to a change in the amino acid or nucleotide sequence resulting in the absence of one or more amino acid or nucleotide residues. A deletion may remove at least 1, 2, 3, 4, 5, 10, 20, 50, 100, 200, or more amino acid or nucleotide residues. A deletion of a reference polypeptide may include an internal deletion and/or a terminal deletion (e.g., an N-terminal truncation, a C-terminal truncation or both).

“Insertions” and “additions” in a disclosed protein or nucleotide refers to changes in an amino acid or nucleotide sequence resulting in the addition of one or more amino acid or nucleotide residues. An insertion or addition may refer to 1, 2, 3, 4, 5, 10, 20, 30, 40, 50, 60, 70, 80, 90, 100, 150, 200, or more amino acid or nucleotide residues. A variant or homolog of a disclosed protein may have N-terminal insertions, C-terminal insertions, internal insertions, or any combination of N-terminal insertions, C-terminal insertions, and internal insertions.

A “substitution” in a disclosed protein refers to a change of at least one amino acid or nucleotide in a sequence to another amino acid or nucleotide. A substitution may be a single amino acid or nucleotide substitution or more than one amino acid or nucleotide. An amino acid substitution may be a conservative change preserving the size and hydrophobicity of the original amino acid or may change either the length of the amino acid side chain or the charge or reactive group associated with the amino acid side chain. A nucleotide substitution may be a conservative change or deleterious change.

Disclosed protein sequence identities may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Therefore, the invention provides for variants of the proteins of the invention. In one embodiment, the amino acid sequence of the ATG1 protein comprises SEQ ID NO: 2 or a sequence with at least 70%, 75%, 80%, 85%, 90%, 95%, 96%, 97%, 98% or 99% sequence identity to SEQ ID NO: 2; the amino acid sequence of the ATG9 protein comprises SEQ ID NO: 4 or a sequence with at least 70%, 75%, 80%, 85%, 90%, 95%, 96%, 97%, 98% or 99% sequence identity to SEQ ID NO: 4; the amino acid sequence of the ATG12 protein comprises SEQ ID NO: 6 or a sequence with at least 70%, 75%, 80%, 85%, 90%, 95%, 96%, 97%, 98% or 99% sequence identity to SEQ ID NO: 6; the amino acid sequence of the ATG8a protein comprises SEQ ID NO: 8 or a sequence with at least 70%, 75%, 80%, 85%, 90%, 95%, 96%, 97%, 98% or 99% sequence identity to SEQ ID NO: 8; and/or the amino acid sequence of the bZiP87 protein comprises SEQ ID NO: 10 or a sequence with at least 70%, 75%, 80%, 85%, 90%, 95%, 96%, 97%, 98% or 99% sequence identity to SEQ ID NO: 10. In another embodiment, the amino acid sequence of the protein is selected from the group consisting of SEQ ID NO: 2, SEQ ID NO: 4, SEQ ID NO: 6, SEQ ID NO: 8; SEQ ID NO: 10, sequences having at least 95% identity to one of SEQ ID NO: 2, 4, 6, 8, 10, and combinations thereof.

The invention also provides for variants of the polynucleotide sequences encoding the proteins described herein. The polynucleotide sequence encoding ATG1 may comprise SEQ ID NO: 1 or a sequence with at least 70%, 75%, 80%, 85%, 90%, 95%, 96%, 97%, 98% or 99% sequence identity to SEQ ID NO: 1; the polynucleotide sequence encoding ATG9 may comprise SEQ ID NO: 3 or a sequence with at least 70%, 75%, 80%, 85%, 90%, 95%, 96%, 97%, 98% or 99% sequence identity to SEQ ID NO: 3; the polynucleotide sequence encoding ATG12 may comprise SEQ ID NO: 5 or a sequence with at least 70%, 75%, 80%, 85%, 90%, 95%, 96%, 97%, 98% or 99% sequence identity to SEQ ID NO: 5; the polynucleotide sequence encoding ATG8a may comprise SEQ ID NO: 7 or a sequence with at least 70%, 75%, 80%, 85%, 90%, 95%, 96%, 97%, 98% or 99% sequence identity to SEQ ID NO: 7; and/or the polynucleotide sequence encoding bZiP87 may comprise SEQ ID NO: 9 or a sequence with at least 70%, 75%, 80%, 85%, 90%, 95%, 96%, 97%, 98% or 99% sequence identity to SEQ ID NO: 9. In some embodiments, the polynucleotide sequence encoding the protein is selected from the group consisting of SEQ ID NO: 1, SEQ ID NO: 3, SEQ ID NO: 5, SEQ ID NO: 7, SEQ ID NO: 9, sequences having at least 95% identity to one of SEQ ID NO: 1, 3, 5, 7, 9, and combinations thereof. Those of skill in the art are aware of the degeneracy of the genetic code and that different organisms have preferred codon utilization. Thus the polynucleotides presented herein may be codon optimized for use in other related species.

The plant cell may further comprise a second polynucleotide. The second polynucleotide may encode any of the other autophagy related proteins noted above or may encode an ATG8a protein. The second polynucleotide may be operably connected to a second heterologous promoter. In some embodiments, the amino acid sequence of the ATG8a protein comprises SEQ ID NO: 8 or a sequence with at least 70%, 75%, 80%, 85%, 90%, 95%, 96%, 97%, 98% or 99% sequence identity to SEQ ID NO: 8.

“Percentage of sequence identity” is determined by comparing two optimally aligned sequences over a comparison window. The aligned sequences may comprise insertions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100. Protein and nucleic acid sequence identities can be evaluated using the Basic Local Alignment Search Tool (“BLAST”), which is well known in the art (Karlin and Altschul, Methods for assessing the statistical significance of molecular sequence features by using general scoring schemes. Proc. Natl. Acad. Sci. USA (1990) 87:2267-2268; Altschul et al. Gapped BLAST and PSI-BLAST: a new generation of protein database search programs. Nucl. Acids Res. (1997) 25:3389-3402). The BLAST programs identify homologous sequences by identifying similar segments between a query amino acid or nucleic acid sequence and a test sequence, which is preferably obtained from a protein or nucleic acid sequence database. The BLAST programs can be used with the default parameters or with modified parameters provided by the user.

In the Examples, the inventors demonstrate that overexpression of autophagy proteins or their regulators, or combinations thereof, increases autophagy in plants. The increase in autophagy may be phenotypically identified as increasing biomass, yield, stress tolerance and heat tolerance of the plant or may allow the plant to require less fertilizer to grow. Thus, the present invention provides methods of producing a “genetically modified” plant and a genetically modified plant produced by said methods. The invention also provides plants comprising the genetically modified cells. The term “plant” is used broadly herein to refer to a plant at any stage of development or a part of a plant, including a plant cutting, a plant cell, a plant cell culture, a plant organ, a plant tissue, or a plantlet. Particularly useful parts of a plant include harvestable parts and parts that can be used for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

A “plant cell” is the basic structural unit of the plant, comprising a protoplast and a cell wall. A plant cell can be in the form of an isolated single cell or aggregate of cells (e.g., a friable callus or a cultured cell) or can be part of a higher organized unit. The cell or tissue culture will preferably be capable of regenerating plants. In some embodiments, the plant cell is a Zea mays cell. In other embodiments, the plant cell is a sorghum cell, a millet cell, a rice cell, a wheat cell, a soy cell, a tomato cell, or a cotton cell.

The terms “genetically modified”, “genetically engineered”, “transformed” and “transgenic” are used interchangeably. Similarly, the term “genetic transformation” is used interchangeably with “genetic modification”. As used herein, the terms “modified” or “modifying” refer to using any laboratory methods available to those of skill in the art including, without limitation, genetic engineering techniques (i.e. CRISPR/Cas gene editing techniques or Recombinant DNA/transgenic technologies), traditional breeding/selection techniques, or forward genetic techniques to affect the enzyme activity or expression of a protein in a plant cell. Plant cells may be genetically modified using techniques available to those of skill in the art including agrobacterium mediated transformation, transfection, transduction, electroporation or any other means of introducing nucleic acids into a plant cell. The nucleic acids introduced may be DNA or RNA and should be introduced in such a way as to allow expression of proteins encoded by the nucleic acids. The methods also include plants engineered to have more than one copy of the native gene linked to the native promoter or a heterologous promoter which may be engineered to through a means such as CRISPR based genetic editing to overexpress a native protein.

As used herein, “enzyme activity” refers to the ability of a disclosed protein to catalyze a particular chemical conversion. The enzyme activity of a disclosed protein may be increased by at least 5%, 10%, 20%, 30%, 40%, 50%, 55%, 60%, 65%, 70%, 75%, 80%, 85%, 90%, 95%, or more as compared to a control plant cell. As used herein, a “control plant” or a “control plant cell” is a plant or cell that has not been modified as described herein. Exemplary control plants and cells may include those from a natural or wild-type plant species and may be a plant of the same variety or cultivar as the particular plant being modified. The term “expression” may refer either to the levels of an RNA encoding a disclosed protein in a cell or the levels of the disclosed protein in a cell. The expression of the disclosed protein is increased by at least 5%, 10%, 20%, 30%, 40%, 50%, 55%, 60%, 65%, 70%, 75%, 80%, 85%, 90%, 95%, or more as compared to a control plant cell in the methods of increasing the expression of the ATG1, ATG9, ATG12, ATG8a, bZIP87 proteins as described here. In a further embodiment, the expression or enzyme activity of the at least one protein is increased as compared to a control plant cell by at least 10%.

### Constructs:

The genetically modified plants may include a construct or expression cassette, which may be integrated into the plant chromosome or maintained extra-chromosomally. The construct or expression cassette may comprise a native or a heterologous promoter operably connected to a polynucleotide encoding at least one protein selected from the group consisting of ATG1, ATG9, ATG12, ATG8a, bZIP87, and combinations thereof. The construct or expression cassette may also include a second promoter operably connected to a polynucleotide encoding an ATG8a polypeptide or any of the other polypeptides including ATG1, ATG9, ATG12 or bZIP87.

As used herein, the term “construct” refers to a recombinant polynucleotide, i.e., a polynucleotide that was formed by combining at least two polynucleotide components from different sources, natural or synthetic. For example, a construct may comprise the coding region of one gene operably linked to a promoter to allow for expression of a protein. The combination of a promoter operably connected to a gene such that a protein is made from the construct after introduction into a cell is an “expression cassette”. An expression cassette may be part of a construct and may be maintained extra-chromosomally or may be integrated into the chromosome via traditional homologous recombination or via Crispr/Cas or a related form of gene editing. The polynucleotides encoding proteins may encode a protein that is (1) found within the same genome, (2) from the genome of a different species, or (3) synthetic. A construct may comprise a marker gene or selectable marker gene operably linked to a promoter or the linked in frame to the coding region of the polynucleotide. As used herein, the term “marker gene” refers to a gene that encodes a marker protein that has a detectable phenotype in the plant, or part thereof. Exemplary marker genes include yellow fluorescent protein (YFP), mCherry, or any other marker known to those of skill in the art. The construct may also include a selectable marker such that cells containing the marker have a survival advantage. The promoter used in the constructs and expression cassettes may be a native promoter for the polynucleotide to which it is operably connected or a heterologous promoter. Constructs can be generated using conventional recombinant DNA methods.

The terms “polynucleotide,” “nucleic acid,” and “oligonucleotide” are used interchangeably to refer a polymer of DNA or RNA. A polynucleotide may be single-stranded or double-stranded and may represent the sense or the antisense strand. A polynucleotide may be synthesized or obtained from a natural source. The polynucleotide may be all or part of a gene sequence, including the upstream and/or downstream regulatory elements, signal peptides, introns or may include only a cDNA or may be codon optimized when a polynucleotide from one organism is used for expression of a protein in a distinct organism. In some embodiments, the plant cell comprises a polynucleotide selected from the group consisting of ATG1 (SEQ ID NO: 1), ATG9 (SEQ ID NO: 3), ATG12 (SEQ ID NO: 5), ATG8a (SEQ ID NO: 7), bZIP87 (SEQ ID NO: 9), and combinations thereof. In another embodiment, the plant cell further comprises a second polynucleotide encoding a second autophagy protein. The second autophagy protein may be an ATG8a protein. The second polynucleotide is operably connected to a second promoter. In a further embodiment, the second polynucleotide encodes ATG8a (SEQ ID NO: 8) or other combinations of ATG1, ATG9, ATG12 and bZIP87.

The term “promoter” refers to a DNA sequence that regulates the expression of a gene. Typically, a promoter is a regulatory region that is capable of binding RNA polymerase and initiating transcription of a downstream (3′ direction) sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA. A promoter is “operably linked” or “operably connected” to a polynucleotide if the promoter is connected to the polynucleotide such that it can affect transcription of the polynucleotide. It is understood by those skilled in the art that different promoters may direct the expression of a gene in different tissues or cell types, at different stages of development, or in response to different environmental conditions. Suitable promoters for use with the present invention include, but are not limited to, constitutive, inducible, temporally regulated, developmentally regulated, chemically regulated, tissue-preferred, and tissue-specific promoters. The promoter may be a native promoter. As used herein, the term “native” refers to a promoter that is endogenous to the plant and drives expression of the natively associated gene. Native promoters may be modified by CRISPR-Cas9 technologies to enhance their expression and/or the expression of the genes they regulate.

The term “heterologous promoter” is defined as a promoter that is not operably connected to the wild-type gene in the plant, i.e. the promoter is not natively associated with the gene. The heterologous promoter may be a plant promoter or may be derived from a virus or other organism. Suitable promoters for expression in plants include, without limitation, the 35S promoter of the cauliflower mosaic virus, ubiquitin (Ubi), bundle sheath promotor, tCUP cryptic constitutive promoter, the Rsyn7 promoter, pathogen-inducible promoters, the maize In2-2 promoter, the tobacco PR-1a promoter, glucocorticoid-inducible promoters, Act promoters, estrogen-inducible promoters and tetracycline-inducible and tetracycline-repressible promoters. Those of skill in the art are familiar with a wide variety of additional promoters for use in various cell or tissue types. In the Examples, the heterologous maize promoter used was the Ubiquitin-1 (ZmUbi1) promoter.

### Methods of Making the Plants and Plant Cells

It will be apparent to one of ordinary skill in the art that there are multiple potential ways to increase the activity or expression of the disclosed proteins in a plant or plant cells to generate plants with an increase in at least one phenotypic trait. These include, but are not limited to, modifying the gene encoding any one of these proteins by, for example, introducing targeted mutations; modifying a mRNA (or levels thereof) encoding any one of these proteins using, for example, transgenic techniques; or by enhancing the activity of the disclosed proteins at the protein level. One method of generating a genetically modified plant comprises introducing an expression cassette into plant cells. The expression cassette may be part of a construct and may be introduced into plant cells via any method available to those of skill in the art. In the Examples, the expression cassette is introduced by transforming an explant with a DNA construct comprising a heterologous maize promoter operably connected to a polynucleotide sequence encoding at least one autophagy protein selected from Autophagy-related protein 1 (ATG1), Autophagy-related protein 9 (ATG9), Autophagy-related protein 12 (ATG12), Autophagy-related protein 8a (ATG8a), bZIP transcription factor 87 (bZIP87), or combinations thereof. The expression cassette may optionally include a marker gene, and/or a selectable marker. Inclusion of a marker gene or selectable marker allows one to contact the explant with a selective agent and select explants or select cells expressing the marker which indicates that the introduction of the polynucleotide was successful. Finally, the explant or cells are regenerated to produce a transformed plant from the transgenic cells. The marker gene may be fused to the polynucleotide sequence to join the genes such that they are transcribed and translated as a single unit, producing a single fusion polypeptide, but a marker-less protein may also be used.

As used herein, the term “explant” refers to a cell, organ, or piece of tissue from a plant that has been transferred to a nutrient medium. Explants are the target material for transformation. They comprise meristematic tissue, which consists of undifferentiated cells that give rise to multiple plant structures (e.g., stems, roots, leaves, germline tissue, and seeds). Plant tissues that can be used as explants include, without limitation, embryos, cotyledons, hypocotyls, leaf bases, mesocotyls, plumules, protoplasts, and embryonic axes. A plant cell can be in the form of an isolated single cell or aggregate of cells (e.g., a friable callus or a cultured cell) or can be part of a higher organized unit. The cell or tissue culture will preferably be capable of regenerating plants.

After the explants are transformed with the construct, they may be cultured/grown in the presence of a selective agent. As used herein, the term “selective agent” refers to a compound that terminates or retards the growth of most of the plant cells into which the construct has not been delivered. Thus, a selective agent permits differentiation between cells that were successfully transformed with the construct and cells that were not transformed. Exemplary selective agents include, but are not limited to, kanamycin, hygromycin, streptomycin, chloramphenicol, ampicillin, erythromycin, spectinomycin, tetracycline, bialaphos, glyphosate, glufosinate, rifampicin, or dicamba, and the like. For example, in some embodiments, the selectable marker is encoded by the aadA1a gene and the selective agent is spectinomycin. In other embodiments, the selectable marker is encoded by the hra gene and the selective agent is imazapyr. The transformed explants are then regenerated into plants. The invention thus provides a plant comprising the genetically modified cells of the invention, as well as a seed, part, progeny or asexual propagate of a plant comprising the genetically modified plant cell(s).

In some embodiments, the method comprises introducing an expression cassette. Therefore, the invention provides a method of making an expression cassette and introducing it into the plant cell. An “expression cassette” is a polynucleotide comprising a sequence encoding a polypeptide or a functional RNA as well as elements needed to express the encoded polypeptide or RNA (e.g., a promoter). The sequences controlling the expression of the gene are commonly referred to as a regulatory unit. Most parts of the regulatory unit are located upstream of coding sequence of the gene and are operably linked thereto. The expression cassette may also contain a downstream 3′ untranslated region comprising a polyadenylation site or other terminator sequences. The regulatory unit may be directly linked to the sequence to be expressed or separated therefrom by intervening DNA, e.g., by the 5′-untranslated region of a gene.

The expression cassette may be designed to modify expression of the protein(s) of interest in order to achieve one or more desirable phenotypic traits, such as traits of agronomic or industrial interest. In one embodiment, the expression or activity of the at least one protein is increased as compared to a control plant cell by at least 5%, 10%, 20%, 30%, 40%, 50%, 55%, 60%, 65%, 70%, 75%, 80%, 85%, 90%, 95%, or more as compared to a control plant cell. In another embodiment, the expression or activity of the at least one protein is increased as compared to a control plant cell by at least 10%. Examples of traits of agronomic interest include, but are not limited to, disease, insect, or pest tolerance; herbicide tolerance; quality improvements, such as yield, fiber quality, nutritional enhancements (e.g., oil modification), or improved flavor; environmental or stress tolerances; low fertilizer tolerance; any desirable changes in plant physiology, growth, development, morphology; and plant product production (e.g., starch, biopolymers, pharmaceutical peptides, etc.).

The genetically modified plants described here have at least one phenotypic trait that is distinct from the control plants lacking the genetic modification of increased expression of at least one autophagy protein selected from ATG1, ATG8a, ATG9, ATG12, bZIP87 and combinations thereof. The phenotypic traits are selected from the group consisting of biomass, grain yield, stress tolerance, heat tolerance, and low fertilizer tolerance. As noted above, the phenotypic trait may be increases by at least 10%, 20%, 30%, 40%, 50%, 60%, 70%, 80%, 90% or even more than 100% as compared to a control plant. The increased expression of at least one of the proteins listed may allow the genetically modified plants to be planted in areas that would otherwise not be suitable or commercially viable for the plant. For example, the plant may be planted in areas that experience drought or require large fertilizer inputs and the genetically modified plants may perform better than the control plants when used in these areas.

### Methods of Using the Genetically Modified Plants and Plant Cells

Methods of increasing at least one phenotypic trait of a genetically modified plant are also provided. The methods include modifying a plant to over-express at least one autophagy protein including one of ATG1, ATG8, ATG9, ATG12, bZIP87 as compared to a control plant not modified to over-express these proteins. The methods may include introducing an expression cassette into cells of the plant and growing the plant. The expression cassette may comprise a promoter operably connected to a polynucleotide encoding at least one autophagy protein selected from the group consisting of ATG1, ATG8a, ATG9, ATG12, bZIP87 and combinations thereof. The methods may include genetically editing the plant for over-expression of at least one of ATG1, ATG8, ATG9, ATG12, bZIP87. The phenotypic trait may include increased biomass, yield, stress tolerance, heat tolerance, and reduction in fertilizer input. The genetically modified plant demonstrates an increase in the phenotypic trait as compared to a control plant. The genetically modified plants may include the plant cells expressing the autophagy proteins at an increased level as compared to control plants and plant cells. The plant may be grown from a seed derived from the genetically modified plants provided here.

In the Examples, expression of autophagy proteins and regulators of autophagy was increased in transgenic plants through use of a strong heterologous maize promoter and phenotypic data was collected on the resulting plants, as grown in the greenhouse or the field. Specific phenotypic traits collected on the transgenic plants of the invention include but are not limited to biomass, shoot or stem height or dry weight, chlorophyll content, pollen production, plant height, maturity, days to reproductive maturity, days to tasseling, anthesis silk interval (ASI), grain yield, number of ears per plant, number of kernels per plant or per ear, kernel dry weight, kernel protein and starch content, stress tolerance, heat tolerance, and low fertilizer tolerance. Other phenotypic traits related to yield and stress tolerance are also expected to perform better in the genetically modified plants provided as compared to control plants. In one embodiment, the method of increasing at least one phenotypic trait of a genetically modified plant comprises increasing yield, and the yield is measured by the number or weight of seeds produced by the genetically modified plant. In one embodiment, the phenotypic trait is increased by at least 5%, 10%, 20%, 30%, 40%, 50%, 55%, 60%, 65%, 70%, 75%, 80%, 85%, 90%, 95%, or more as compared to a control plant. In another embodiment, the phenotypic trait is increased by 10-20% in the genetically modified plant as compared to the control plant.

Methods of using the genetically modified plants described herein are also provided. The methods may include planting any one of the plants described herein in an area likely to experience a stress or a risk to the plant. The area may be at risk of drought, heat or other abiotic stress.

Methods of decreasing the amount of fertilizer used to grow plants are also provided. The methods include growing a genetically modified plant that overexpresses at least one of ATG1, ATG8a, ATG9, ATG12, bZIP87 as compared to a control non-modified plant. The plant may comprise a heterologous or native promoter operably connected to a polynucleotide encoding at least one autophagy protein selected from the group consisting of ATG1, ATG8a, ATG9, ATG12, bZIP87 and combinations thereof. The genetically modified plant is provided at least 10, 20%, 30%, 40%, 50% less fertilizer as compared to growing a control plant and similar results may be achieved as compared to a fully fertilized control plant or superior results may be achieved when a control plant is given similar fertilizer amounts.

In one embodiment, the method comprises decreasing the amount of fertilizer applied to the plant during the growing season. The amount of fertilizer used to grow the genetically modified plant to the point of harvest as compared to a control plant grown under the same conditions is reduced by at least 10%, 15%, 20%, 30%, 40%, 50%, 60%, 70%, 80%, 90%, 95% or more. In another embodiment, the method comprises decreasing the amount of fertilizer used to grow the plant. The amount of fertilizer used to grow the genetically modified plant to the point of harvest is 10%, 20%, 25%, 30%, 40%, 50%, 60%, 70%, 80% of the amount used to grow a control plant grown under the same conditions.

The methods may further include applying one or more fertilizers to the plant(s) of the invention or to the area they are planted in. As used herein, the term “fertilizer” refers to any nutrient input applied to the soil that is biologically available to a plant. As used herein, “applying” may be carried out through any of the variety of procedures used to apply fertilizers to plants that will be apparent to the skilled artisan. Suitable application methods may include, without limitation spraying or dusting. Other suitable application procedures can be envisioned by those skilled in the art. In the studies disclosed herein, the inventors grew transgenic maize plants under variable fertilizer treatments to determine if they exhibited improvements in the desired phenotypic traits compared to the control plants. As described in the Examples, the inventors grew the transgenic plants under high Nitrogen (N) (15 mM NO₃⁻) and low N (0.15 mM NO₃⁻) fertilizer application rates in the greenhouse and the field. The inventors discovered that the genetically modified plants exhibited increased biomass, yield, and resistance to abiotic and biotic stress, as compared to the control plants, under several different fertilizer application rates. The inventors observed an increase in beneficial phenotypic traits even under lower Nitrogen application rates. Thus, in some embodiments, the method comprises reducing nitrogen application by 10% when growing the genetically engineered plant as compared to a control plant.

Methods of generating plants with increased stress tolerance or yield are provided. The methods include growing a genetically modified plant that overexpresses at least one of ATG1, ATG8a, ATG9, ATG12, bZIP87 and the stress tolerance and yield are increased as compared to a control non-modified plant. The plant may comprise a promoter operably connected to a polynucleotide encoding at least one of ATG1, ATG8a, ATG9, ATG12, and bZIP87. The genetically modified plant may have increased stress tolerance and/or increased yield as compared to a control plant. The stress tolerance may include heat, drought, pests, disease, or other forms of stress.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

### Development of Transgenic Lines

In the following example, the inventors describe the development of transgenic lines overexpressing maize Atg8a (SEQ ID NO: 7) and Atg9 (SEQ ID NO: 3) fused to fluorescent proteins, Yellow Fluorescent Protein (YFP) and mCherry, respectively.

The plasmid pMCG1005-YFP-Atg8a expressing YFP fused to the N terminus of wild-type maize Atg8a (Zm00001eb105850) and expressed under the control of the heterologous maize Ubiquitin-1 (ZmUbi1) promoter were transformed into maize line HI-II. YFP-Atg8a plants expressing strong YFP signals were backcrossed at least four times into maize W22 inbred background (Li, F., Chung, T., Pennington, J. G., Federico, M. L., Kaeppler, H. F., Kaeppler, S. M. et al. (2015) Autophagic recycling plays a central role in maize nitrogen remobilization Plant Cell 27, 1389-1408 10.1105/tpc.15.00158).

The plasmid pRC7771-mCherry-Atg9 expressing mCherry fused to the C terminus of wild-type maize Atg9 (Zm00001eb010620) and expressed under the control of the ZmUbi1 promoter was transformed into the LH244 inbred line. The T0 mCherry-Atg9 plants expressing strong mCherry signals were self-crossed (LH244 inbred background). A plasmid expressing only mCherry was used as a control.

Results from Growth Assays:

### 1. Nitrogen (N) Treatments in Greenhouse Conditions:

YFP-Atg8a-overexpressing maize plants were grown in pots (0.5 L; 12 cm diameter and 12 cm height), typically filled up to 11 cm with a well-rinsed (>20 volumes) Pro-line Germinating Mix (Jolly Gardener)/Vermiculite 2:1 soil mixture, in a growth chamber under a 16 h light/8 h dark photoperiod and day/night temperatures of 27° C./21° C.

The plants were grown for 2 weeks at 25° C. and fertilized twice a week with modified Hoagland solution containing 15 mM N. Two weeks after planting, the pots were washed with distillated water and fertilized with 50 ml of 15 mM (high N treatment) or 0.15 mM N (low N treatment), for two weeks (4 applications total). At the end of the 4-week experiment, we measured the shoot fresh weight and total chlorophyll content in the 2^{nd} leaf.

The solution used for the low N treatment contained 0.15 mM KNO₃, 0.1 mM CaCl₂), 0.75 mM KHSO₄, 0.25 mM KH₂PO₄, 0.65 mM MgSO₄, 0.1 mM Fe-EDTA, 0.01 mM boric acid, 0.005 mM NH₄tetramolybdate, and micronutrients (1 mM MnSO₄, 1 mM ZnSO₄, 0.5 mM CuSO₄). For the high-N treatment, the solution was adjusted by adding KNO₃ to a final concentration of 15 mM NO₃.

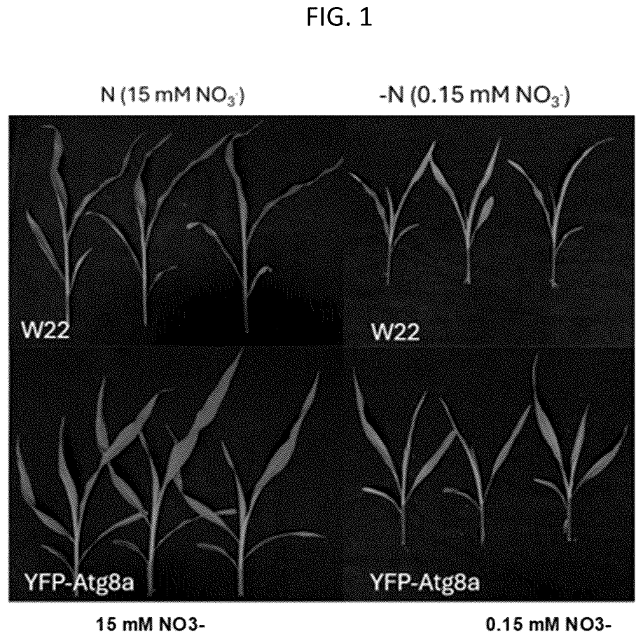

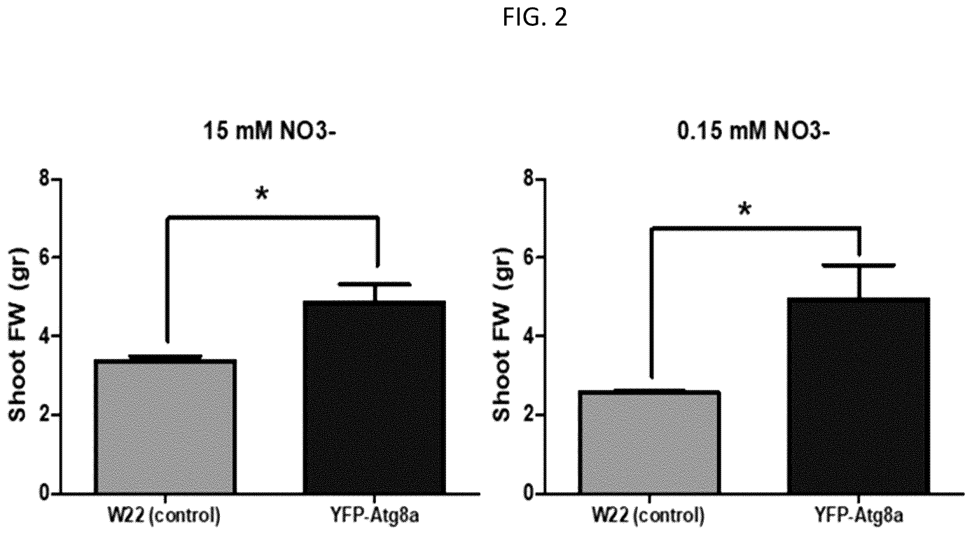

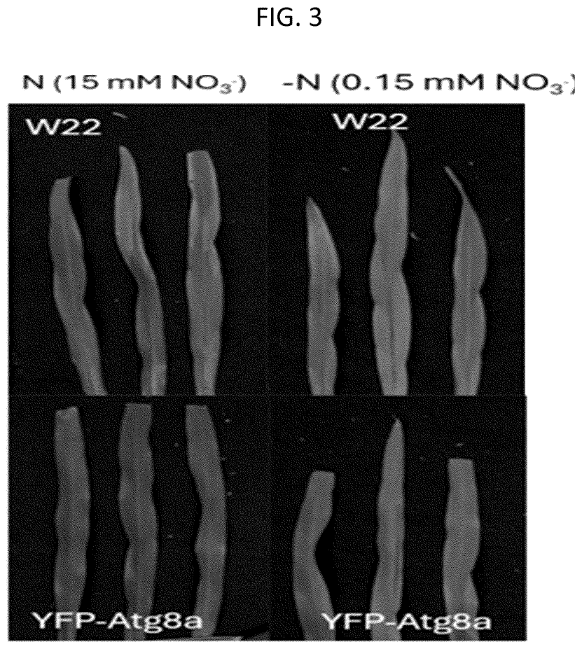

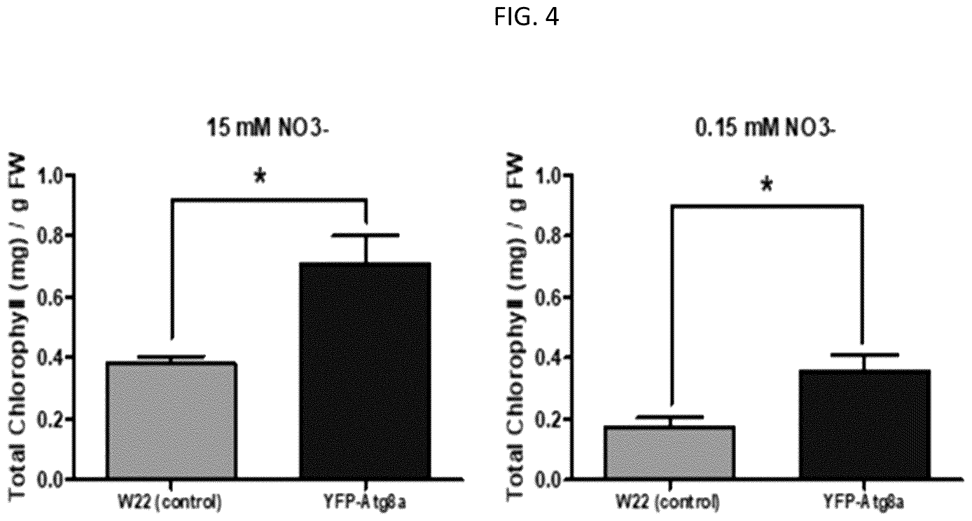

We found that the YFP-Atg8a seedlings produced 50-60% more biomass (shoot fresh weight) and ~50% more total chlorophyll under both N-high and N-low conditions than their wild type (W22) controls (FIG. 1, FIG. 2, FIG. 3, and FIG. 4).

### 2. Field Trials:

To assess the phenotype and yield for field-grown maize, the lines overexpressing YFP-Atg8a and mcherry-Atg9 and their corresponding controls were grown at the West Madison Agricultural Research Station during summer 2024 under well fertilized soil conditions (150-175 kg N per ha. equivalent to 15 15 mM NO₃⁻). Phenological parameters, shoot biomass, total chlorophyll content, pollen production and grain yield were analyzed.

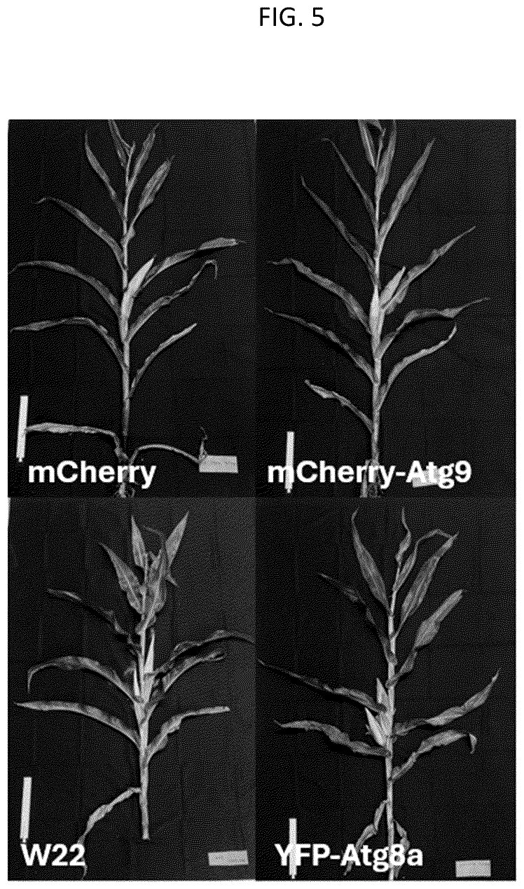

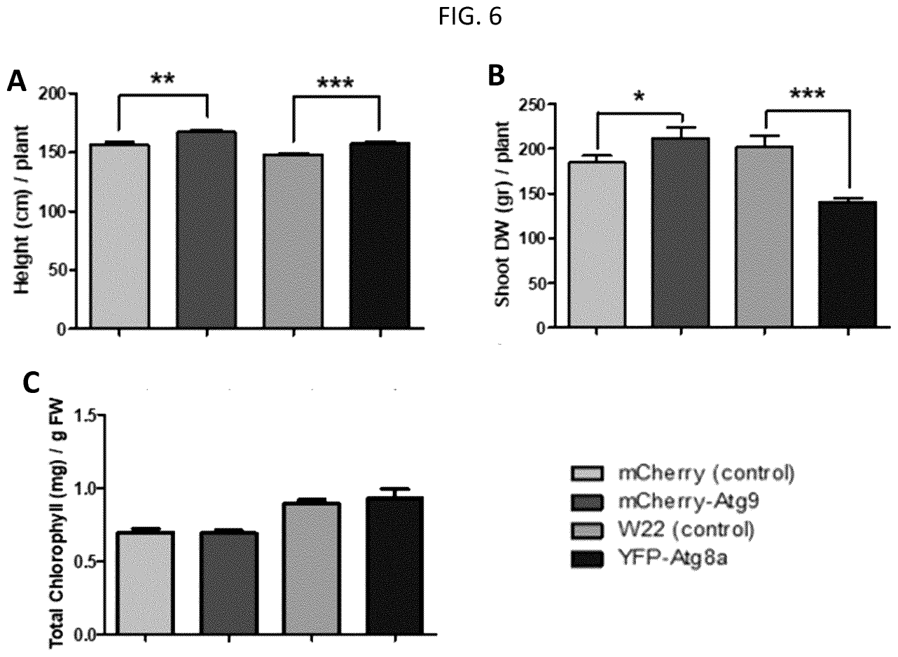

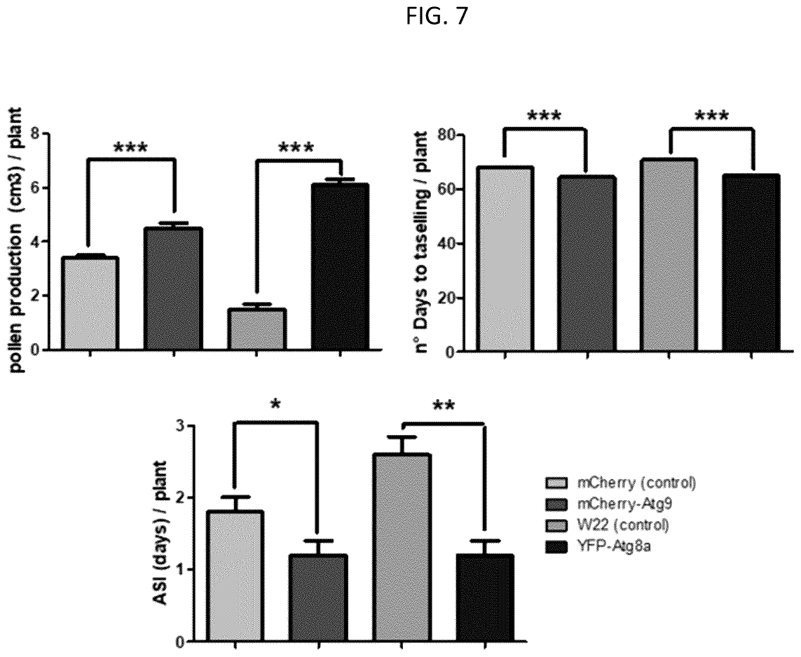

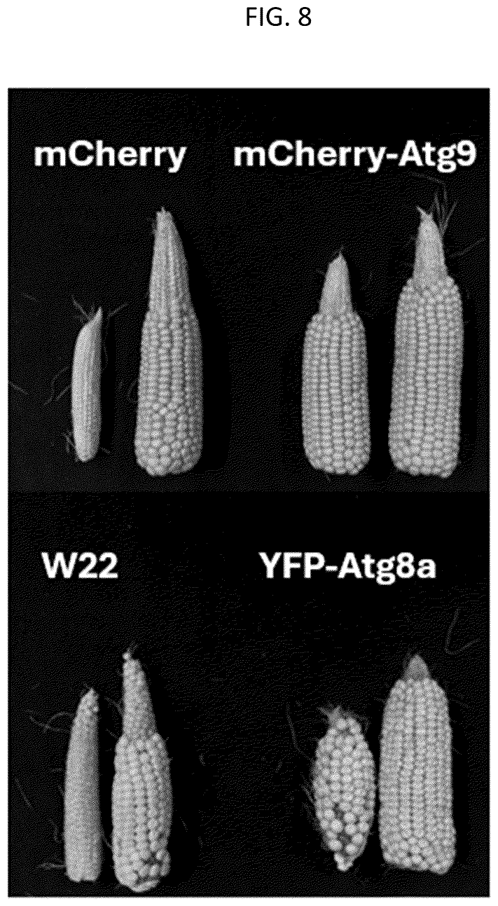

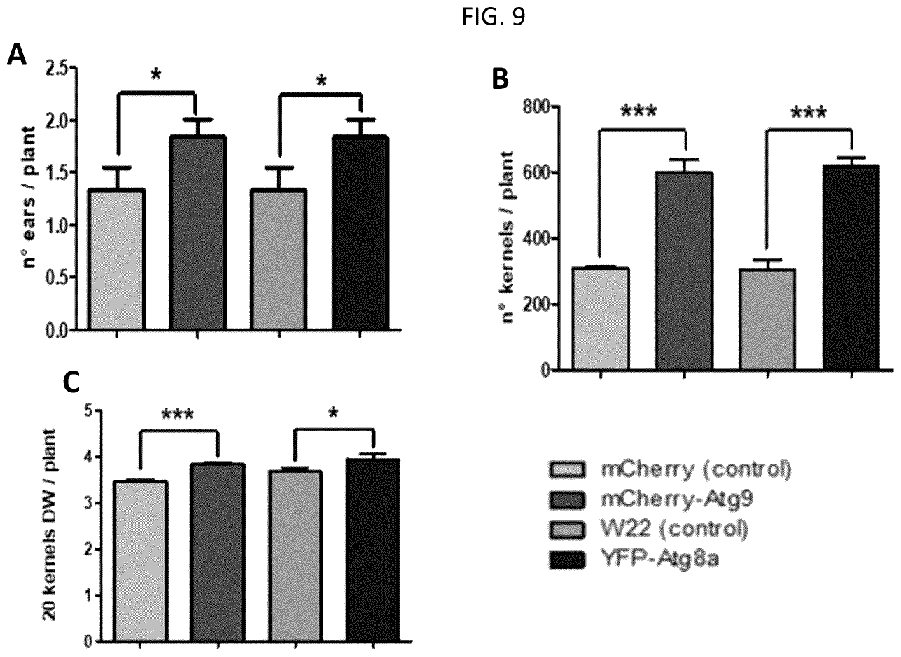

Both the mCherry-Atg9 and YFP-Atg8a transgenic lines 1) grew taller than their controls (FIG. 5 and FIG. 6), 2) produced more ears, 3) produced more pollen, 4) reached the reproductive stage earlier, 5) showed a shorter ASI (the period between pollen release and stigma formation) (FIG. 7), and 6) showed increased grain yield. Indeed, the mCherry-Atg9 expressing lines produce on average 1.9 times more grain (kernels) than transgenic control lines expressing only mCherry, while YFP-Atg8 plants produce 2.0 times more grain than their wild-type counterparts (W22) (FIG. 8 and FIG. 9). This means that in both inbred backgrounds, LH244 and W22, overexpression of a single autophagy gene can double the grain yield per plant under field conditions.

### Example 2

### Development of Transgenic Lines:

In the following example, the inventors describe the development of transgenic lines that overexpress the maize Atg8a (SEQ ID NO: 7) and Atg9 (SEQ ID NO: 3) genes fused to either yellow fluorescent protein (YFP) or mCherry, respectively. Wild type plants or transgenic lines expressing mCherry alone were used as controls.

The plasmid pMCG1005-YFP-Atg8a, which expresses YFP fused to the N-terminus of the wild-type maize Atg8a protein (Zm00001eb105850) and whose expression is controlled by the maize Ubiquitin-1 promoter (ZmUbi1), was transformed into the HI-II background. YFP-Atg8a plants exhibiting a strong YFP signal were backcrossed at least four times with the W22 inbred line (Li et al., 2015).

The plasmid pRC7771-mCherry-Atg9, which expresses mCherry fused to the C-terminus of the wild-type maize Atg9 protein (Zm00001eb010620) and whose expression is controlled by the ZmUbi1 promoter, was transformed into the LH244 inbred line. TO mCherry-Atg9 plants exhibiting a strong mCherry signal were self-pollinated. Same plasmid but with only mCherry (no Atg9) was transformed into the LH244 inbred were generated to use as negative control.

Results from Growth Assays:

### 1. Field Trials:

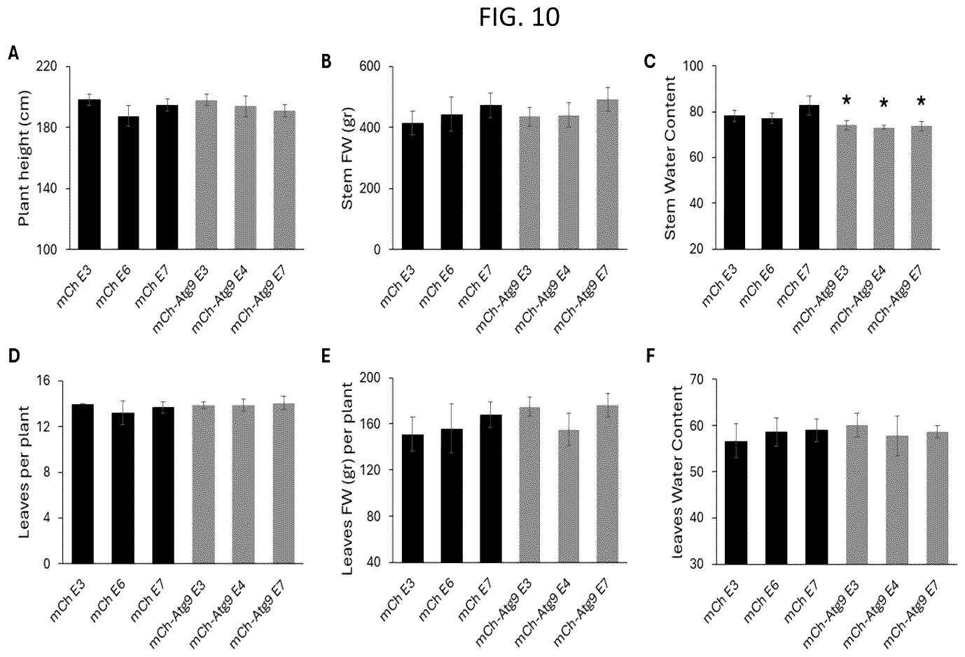

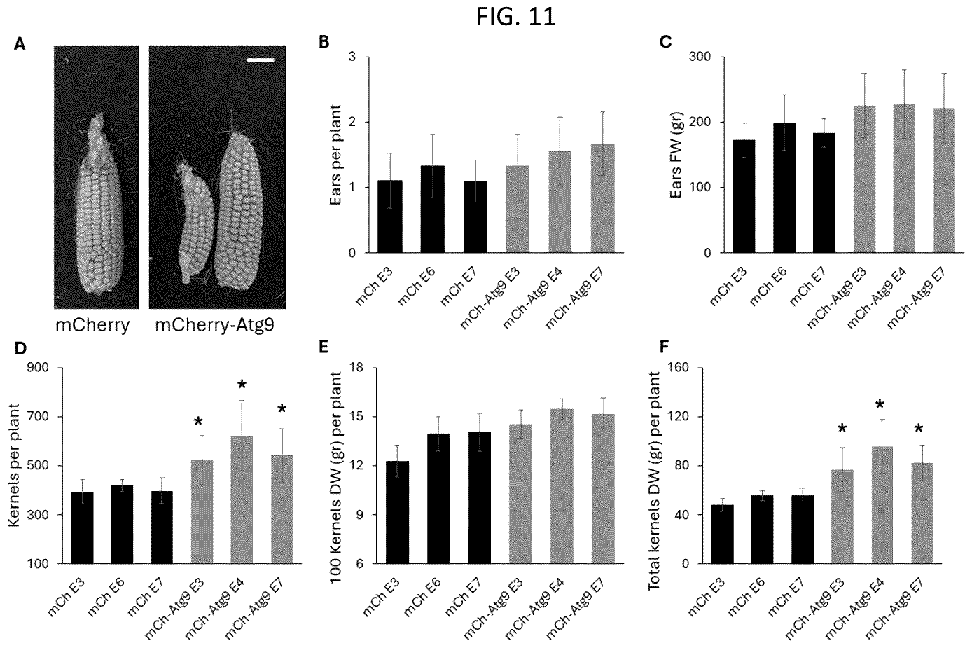

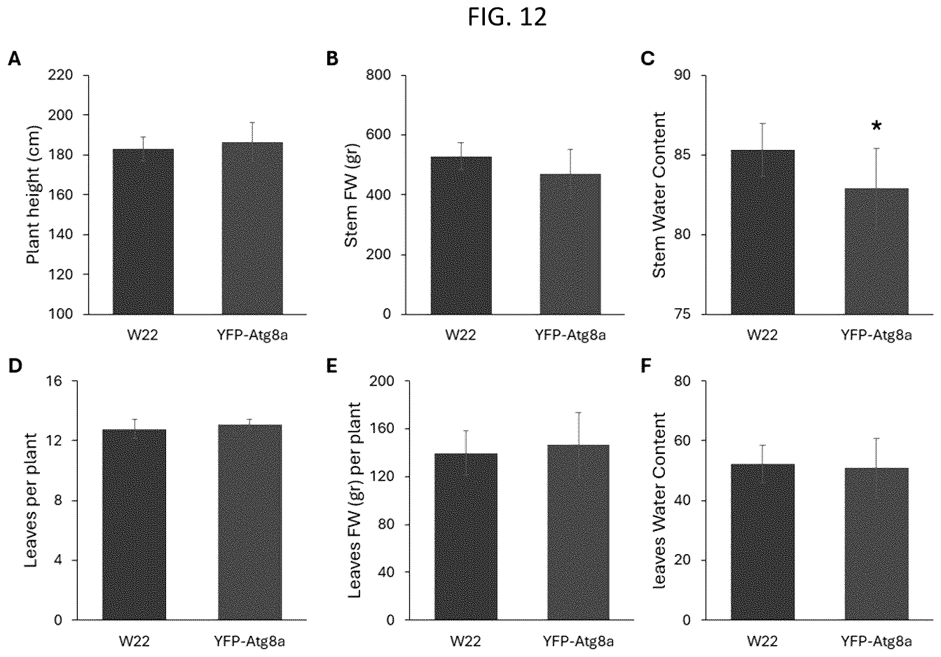

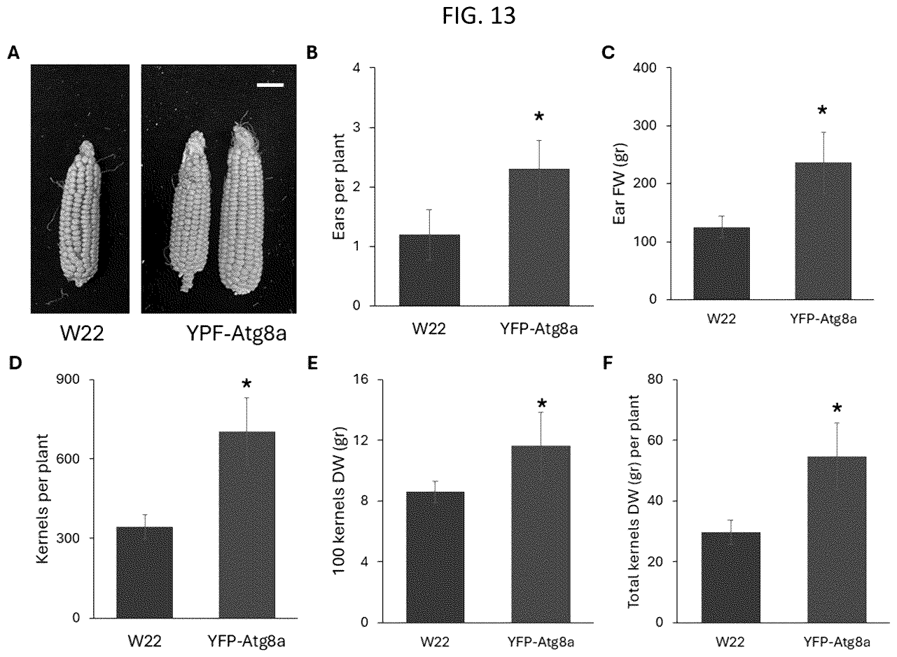

To assess the phenotype and yield for field-grown maize, the lines overexpressing YFP-Atg8a and mcherry-Atg9 as well as their corresponding controls (Wild type W22 inbred line for YFP-Atg8a and mCherry alone for mCherry-Atg9) were grown at the West Madison Agricultural Research Station during the summer 2025, under well fertilized soil conditions. Phenological parameters such as shoot biomass and grain yield were analyzed (FIGS. 10-13). Both the mCherry-Atg9 and YFP-Atg8a transgenic lines 1) Produce more stem dry matter (expressed as FW and less water content; FIG. 10C and FIG. 12C). 3) Show increases kernel yield, due both to an increase in the number of kernels per ear (FIG. 11D and FIG. 13D), and an increase in the dry weight (gr) of the kernel per plant (FIG. 11F and FIG. 13F). Indeed, the lines overexpressing mCherry-Atg9 showed an average total grain dry weight yield 1.4 times higher than the transgenic control lines expressing mCherry alone, while the YFP-Atg8 plants produced an average of 1.8 times more grain dry weight than their wild-type counterparts (FIG. 11 and FIG. 13). This means that, in both genetic backgrounds, LH244 and W22, overexpression of a single autophagy gene can increase 50% to 80% grain yield per plant under field conditions.

### 2. Nitrogen (N) Treatments in Greenhouse Conditions:

Maize plants overexpressing YFP-Atg8a and mCherry-Atg9 were grown in 3.5-gallon pots filled with vermiculite to 85% capacity. The pots were watered until saturated 10 times to ensure the removal of any residual nutrients. The control treatment consisted of watering plants with liquid fertilizer twice a week and only once a week for the nitrogen starvation treatment, with the second watering consisting of plain water. The greenhouse room conditions were: 16-hour light/8-hour dark photoperiod and day/night temperatures of 27° C./21° C. Data on plant height, leaf area, chlorophyll content (measured using a SPAD meter on the 10th leaf), leaf area measurements were taken at 45, 65, and 85 days after planting (DAP) for mCherry-Atg9 and at 65 and 85 DAP for YFP-Atg8a. Pollen production (measured in volume; cm³) was recorded at 85 DAP, while the number of ears produced was recorded 15 days after pollination.

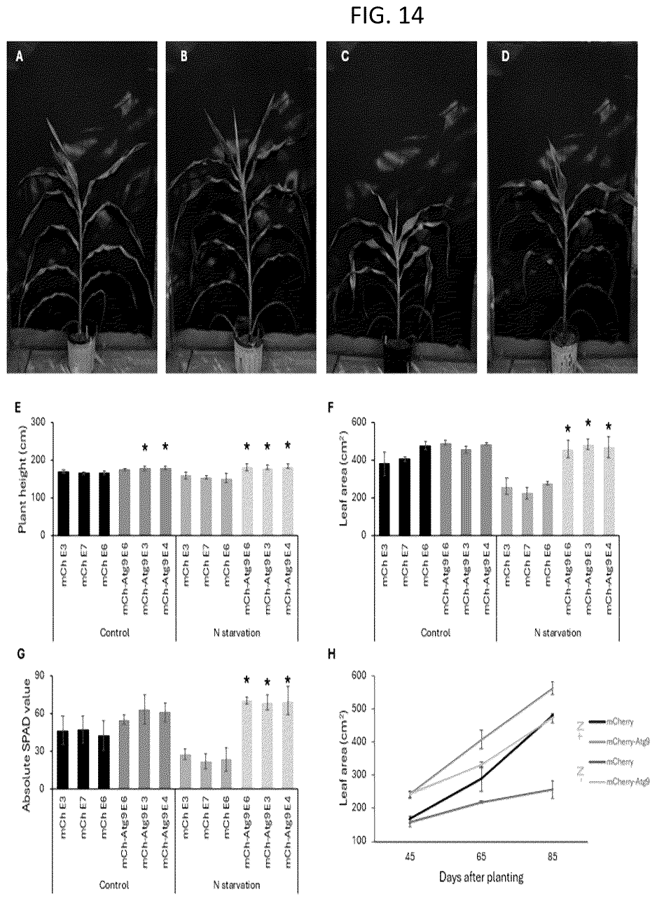

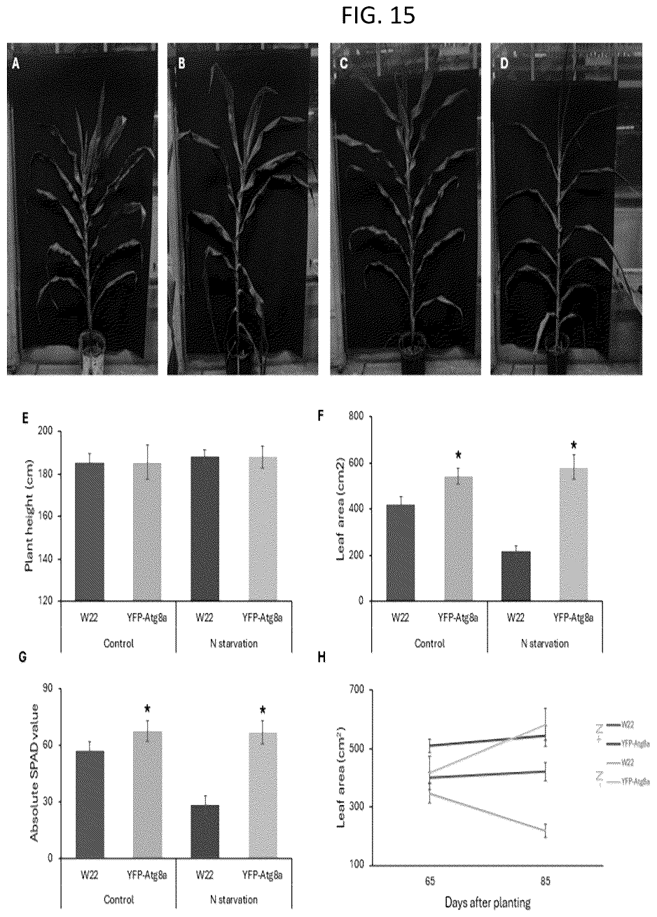

The collected data shows that plants overexpressing mCherry-Atg9 and YFP-Atg8a are less affected than their control mCherry and W22 (FIGS. 14 and 15), after nitrogen starvation treatments. Atg-OE plants are not affected in terms of leaf area and chlorophyll content when exposed to low nitrogen conditions (FIG. 14F, FIG. 14G, FIG. 15F, and FIG. 15G), and have shown better responses on leaf area over time compared to their controls (FIG. 14H and FIG. 15H).

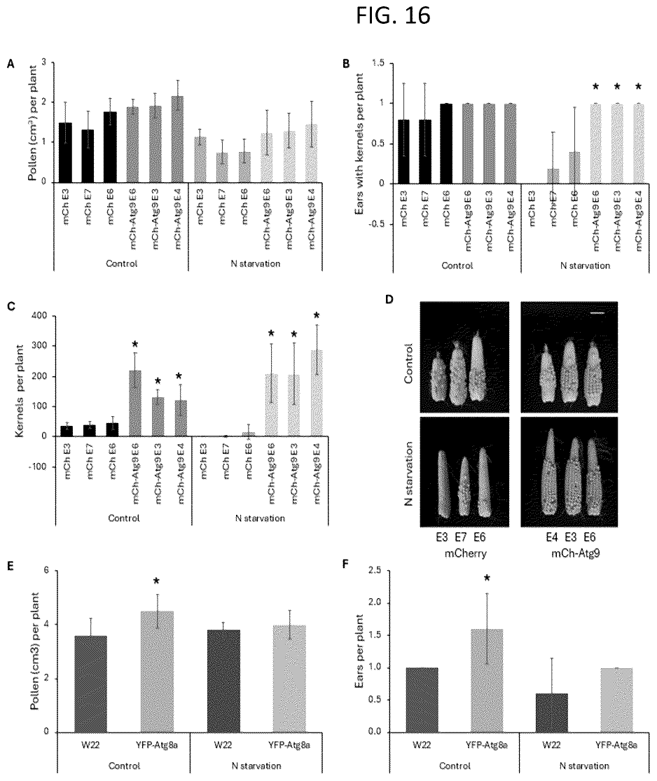

The improvement in biomass production and chlorophyll content in Atg-OE plants is correlated with higher ear with kernels production (FIG. 16B), with mCherry-Atg9 lines approximately doubling the number of ears compared to its control when exposed to nitrogen starvation, and with an important improvement on the kernel production in both conditions (FIG. 16C).

### 3. Low Nitrogen Treatments in Grow Chamber Conditions for New Atg-OE Lines:

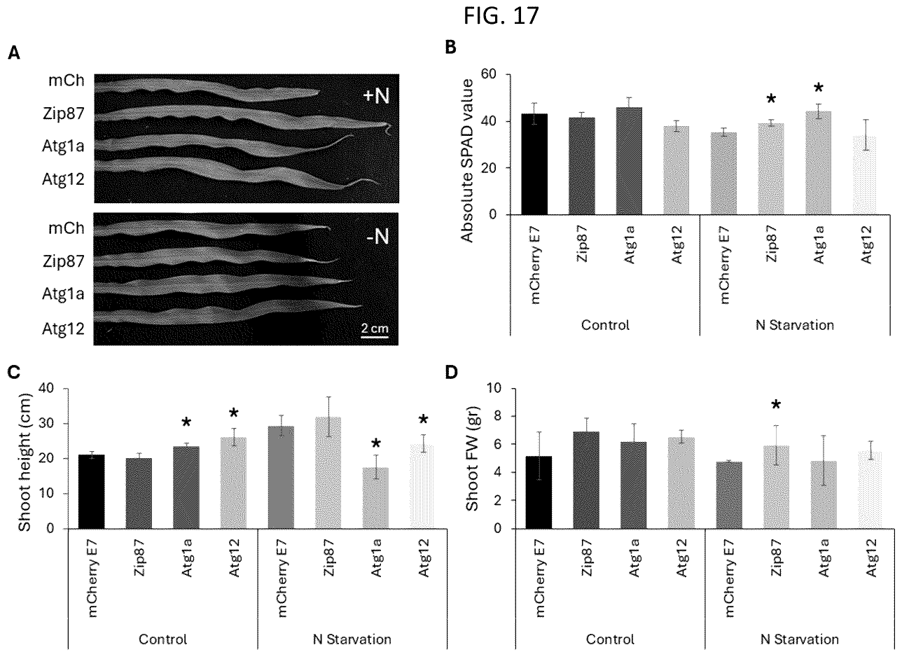

T1 plants overexpressing bZip87, atg1a, and Atg12 were grown under nitrogen starvation using 0.5 L pots (12 cm diameter and 12 cm height) filled up to 11 cm with well-rinsed (>20 volumes) Pro-line Germinating Mix (Jolly Gardener)/vermiculite 2:1 soil under a 16 h light/8 h dark photoperiod and day/night temperatures of 25° C./21° C. Plants were grown for 2 weeks at 25° C. and fertilized twice a week with modified Hoagland solution containing 15 mM N. Then, pots were washed with distilled water and fertilized with 50 ml of 15 mM (high N treatment) or 0.15 mM N (low N treatment), for two weeks (4 applications total). At the end of the 4-week experiment, measurements of shoot heights, shoot FW and total chlorophyll content in the 2nd leaf were taken (FIGS. 17A and 17B). The solution used for the low N treatment contained 0.15 mM KNO₃, 0.1 mM CaCl₂), 0.75 mM KHSO₄, 0.25 mM KH₂PO₄, 0.65 mM MgSO₄, 0.1 mM Fe-EDTA, 0.01 mM boric acid, 0.005 mM NH₄tetramolybdate, and micronutrients (1 mM MnSO₄, 1 mM ZnSO₄, 0.5 mM CuSO₄). For the high-N treatment, the solution was adjusted by adding KNO₃ to a final concentration of 15 mM NO₃.

Compared to their mCherry control, lines overexpressing Atg1a or Atg12 showed increased and decreased shoot height under control and nitrogen deprivation conditions, respectively (FIG. 17C). Plants overexpressing Zip87 contain more chlorophyll and showed increased shoot fresh weight under low nitrogen (FIGS. 17B and 17D). Increased chlorophyll content was also observed in seedlings overexpressing Atg1a after the low-nitrogen treatment compared to the mCherry control (FIG. 17B). As mentioned, these are preliminary results that will be repeated with at least three transformation events and longer growth periods.

### REFERENCES

1. Li, F., Chung, T., Pennington, J. G., Federico, M. L., Kaeppler, H. F., Kaeppler, S. M., Otegui, M. S., and Vierstra, R. D. (2015). Autophagic recycling plays a central role in maize nitrogen remobilization. Plant Cell 27, 1389-1408. doi: 10.1105/tpc.15.00158.

### EMBODIMENTS

1. A genetically modified plant cell comprising a heterologous promoter operably connected to a polynucleotide encoding at least one protein selected from the group consisting of ATG1, ATG9, ATG12, bZIP87, and combinations thereof.
2. The plant cell of embodiment 1, wherein the amino acid sequence of the protein is selected from the group consisting of SEQ ID NO: 2, SEQ ID NO: 4, SEQ ID NO: 6, SEQ ID NO: 10, sequences having at least 95% identity to one of SEQ ID NO: 2, 4, 6, 10, and combinations thereof.
3. The plant cell of embodiment 1 or 2, wherein the plant cell further comprises a second polynucleotide encoding an ATG8 protein, wherein the second polynucleotide is operably connected to a second promoter and optionally wherein the amino acid sequence of the ATG8 protein is selected from the group consisting of SEQ ID NO: 8 or sequences having at least 95% identity to SEQ ID NO: 8.
4. The plant cell of any one of embodiments 1-3, wherein the heterologous promoter is selected from the group consisting of a constitutive promoter, an inducible promoter, and a tissue-specific promoter.
5. The plant cell of embodiment 4, wherein the heterologous promoter is Ubiquitin-1.
6. The plant cell of embodiment 4, wherein the heterologous promoter is a bundle sheath promoter.
7. The plant cell of any one of embodiments 1-6, wherein the plant cell is a Zea mays cell, a sorghum cell, a millet cell, a rice cell, a wheat cell, a soy cell, a cotton cell, or a tomato cell.
8. The plant cell of any one of embodiments 1-7, wherein the cell is genetically modified via genetic transformation or gene editing.
9. The plant cell of any one of embodiments 1-8, wherein the expression or protein activity of the at least one protein is increased as compared to a control plant cell by at least 10%.
10. A plant comprising the cell of any one of embodiments 1-9.
11. A seed, part, progeny or asexual propagate of a plant comprising the plant cell of any one of embodiments 1-9.
12. A method of increasing at least one phenotypic trait of a genetically modified plant, comprising introducing an expression cassette into cells of the plant and growing the plant, wherein the expression cassette comprises a promoter operably connected to a polynucleotide encoding at least one autophagy protein selected from the group consisting of ATG1, ATG8, ATG9, ATG12, bZIP87 and combinations thereof, wherein the phenotypic trait is selected from the group consisting of biomass, yield, stress tolerance, heat tolerance, and low fertilizer tolerance, and wherein the genetically modified plant demonstrates an increase in the phenotypic trait as compared to a control plant not over-expressing the autophagy protein.
13. The method of embodiment 12, wherein the plant comprises the genetically modified plant cell of any one of embodiments 1-9.
14. The method of embodiment 12, wherein the plant is the plant of embodiment 10 or is grown from the seed of embodiment 11.
15. The method of any one of embodiments 12-14, wherein the phenotypic trait is increased by 10-20% in the genetically modified plant as compared to the control plant.
16. The method of any one of embodiments 12-15, wherein the phenotypic trait is yield and the yield is measured by the number or weight of seeds produced by the genetically modified plant.
17. The method of any one of embodiments 12-16, further comprising reducing nitrogen application by 10-20% when growing the genetically engineered plant as compared to a control plant.
18. A method of decreasing the amount of fertilizer used to grow plants comprising: growing a genetically modified plant, wherein the plant is genetically modified to overexpress at least one autophagy protein selected from the group consisting of ATG1, ATG8, ATG9, ATG12, bZIP87 and combinations thereof as compared to a control plant and wherein the genetically modified plant is provided at least 10% less fertilizer as compared to the control plant.
19. A method of generating plants with increased stress tolerance or yield comprising growing a genetically modified plant, wherein the plant is genetically modified to overexpress at least one autophagy protein selected from the group consisting of ATG1, ATG8, ATG9, ATG12, bZIP87 and combinations thereof as compared to a control plant, wherein the genetically modified plant has increased stress tolerance as compared to the control plant.
20. The method of embodiment 19, wherein the stress tolerance is selected from the group consisting of increasing drought tolerance, pest resistance, heat tolerance, and low fertilizer tolerance.
21. The method of embodiment 19, wherein increased yield is selected from the group consisting of increased biomass, increased seed production, or a decreased fertilizer requirement.
22. The method of any one of embodiments 18-21, wherein the genetically modified plant comprises a heterologous promoter operably connected to a polynucleotide encoding at least one autophagy protein selected from the group consisting of ATG1, ATG8, ATG9, ATG12, bZIP87 and combinations thereof.
23. The method of any one of embodiments 18-22, wherein the amount of fertilizer used to grow the genetically modified plant to the point of harvest as compared to a control plant grown under the same conditions is reduced by at least 20%, 30%, 40%, 50% or more.
24. The method of any one of embodiments 18-23, wherein the amount of fertilizer used to grow the genetically modified plant to the point of harvest is 10% of the amount used to grow a control plant grown under the same conditions.
25. The method of any one of embodiments 18-24, wherein the plant comprises the genetically modified plant cell of any one of embodiments 1-9

What is claimed:

1. A genetically modified plant cell comprising a heterologous promoter operably connected to a polynucleotide encoding at least one protein selected from the group consisting of ATG1, ATG9, ATG12, bZIP87, and combinations thereof.

2. The plant cell of claim 1, wherein the amino acid sequence of the protein is selected from the group consisting of SEQ ID NO: 2, SEQ ID NO: 4, SEQ ID NO: 6, SEQ ID NO: 10, sequences having at least 95% identity to one of SEQ ID NO: 2, 4, 6, 10, and combinations thereof.

3. The plant cell of claim 1, wherein the plant cell further comprises a second polynucleotide encoding an ATG8 protein, wherein the second polynucleotide is operably connected to a second promoter and optionally wherein the amino acid sequence of the ATG8a protein is selected from the group consisting of SEQ ID NO: 8 or sequences having at least 95% identity to SEQ ID NO: 8.

4. The plant cell of claim 1, wherein the heterologous promoter is selected from the group consisting of a constitutive promoter, an inducible promoter, and a tissue-specific promoter.

5. The plant cell of claim 4, wherein the heterologous promoter is Ubiquitin-1.

6. The plant cell of claim 4, wherein the heterologous promoter is a bundle sheath promoter.

7. The plant cell of claim 1, wherein the plant cell is a Zea mays cell, a sorghum cell, a millet cell, a rice cell, a wheat cell, a soy cell, a cotton cell, or a tomato cell.

8. The plant cell of claim 1, wherein the cell is genetically modified via genetic transformation or gene editing.

9. The plant cell of claim 1, wherein the expression or protein activity of the at least one protein is increased as compared to a control plant cell by at least 10%.

10. A plant comprising the plant cell of claim 1.

11. A seed, part, progeny or asexual propagate of a plant comprising the plant cell of claim 1.

12. A method of increasing at least one phenotypic trait of a genetically modified plant, comprising introducing an expression cassette into cells of the plant and growing the plant, wherein the expression cassette comprises a promoter operably connected to a polynucleotide encoding at least one autophagy protein selected from the group consisting of ATG1, ATG8a, ATG9, ATG12, bZIP87 and combinations thereof, wherein the phenotypic trait is selected from the group consisting of biomass, yield, stress tolerance, heat tolerance, and low fertilizer tolerance, and wherein the genetically modified plant demonstrates an increase in the phenotypic trait as compared to a control plant not over-expressing the autophagy protein.

13. The method of claim 12, wherein the phenotypic trait is increased by 10-20% in the genetically modified plant as compared to the control plant.

14. The method of claim 12, wherein the phenotypic trait is yield and the yield is measured by the number or weight of seeds produced by the genetically modified plant.

15. The method of claim 12, further comprising reducing nitrogen application by 10-20% when growing the genetically engineered plant as compared to a control plant.

16. A method of decreasing the amount of fertilizer used to grow plants or increasing the stress tolerance or yield of plants comprising: growing a genetically modified plant, wherein the plant is genetically modified to overexpress at least one autophagy protein selected from the group consisting of ATG1, ATG8a, ATG9, ATG12, bZIP87 and combinations thereof as compared to a control plant.

17. The method of claim 16, wherein the genetically modified plant is provided at least 10% less fertilizer as compared to the control plant to produce similar results.

18. The method of claim 16, wherein the stress tolerance is selected from the group consisting of increasing drought tolerance, pest resistance, heat tolerance, and low fertilizer tolerance.

19. The method of claim 16, wherein increased yield is selected from the group consisting of increased biomass, increased seed production, or a decreased fertilizer requirement.

20. The method of claim 16, wherein the genetically modified plant comprises a heterologous promoter operably connected to a polynucleotide encoding at least one autophagy protein selected from the group consisting of ATG1, ATG8a, ATG9, ATG12, bZIP87 and combinations thereof.


SEQUENCE LISTING (10 sequences)

SEQ ID NO: 1 - DNA, 1617 bp, Zea mays
  source 1..1617: mol_type = other DNA
atggacgaggaggaaagggaggaggtgggagagtacaagctgcaagggttggttgggaggggctcgttcgccgaggtgcaccgtgccgcgcaccgccgcactggcgcgcccgcggcggtcaaggcgatcgaccgccgcctcgtcgacaagcgcgtccacgacggcatcctgcaggagagggagatactcaggagcatcgaccaccccaacatcctccgcctcctcgacaccatcgacaccacgaacatgatgtacctggtgctggagtactgcgacggaggcgacctggacgcgtttctccacaagcacggccggctgccggaggccgtagccaaggacttgatgcggcagctcgcggaagggctgaaggtgctgagggggaggaacatcgtgcaccgggacctcaaaccacagaaccttctcctatcaaccaacggggatgacatcgtattgaaaattggtgactttgggtttgccaggtcgctggtgcatgaaaatttagctgctacaatatgtgggtcgccatattatatggcaccagaaatttggcaagggaaggactatgatgcaaagtcggacttgtggagtgttggtatcattctttttcagctagtcacagggaagttgccattcaccgggagtaatgcttttcagctgcaccaaaatgtcatggctaccgatgatcttaacttcccttcagaaattgaagctgatttatgtcctgattgcattgatttgtgcagaagactcctgcaccgtgatccaaagaagagaataagttttgaagaaatcttcaatcacaagtttttggcaacaacaaggaaatgctatgaatcctgtcatgctgtagaattaagagataactgccagacaatcacctctcctgttgtcctaaaaacaaagtctggaagtattgaatcaaaaaatccgaaagtttttgattcatgggagtggattgaacgagaatatgtgcttgttccggaaaactgtacttctatggaaatgtcttcactcaacatcaagtccacaaaggatgatatggatacaagaacggtcagctatggtaggtccactggcaaagggtctgttcagaaccaaatcagagactttactgacacagctactggtgtacaaagccatggatgtgctcctgtggtcatttcacaggagtcagccactgtggattgtagacgagggaaaccacctgattatattacaagacttcattctctgaatcaatatgttcttgttcttgcggatcttgctcgggaaaagctatctaaaggcctgtacctggaggcgttatcaattgaacttgtattattaactatctggaaagaggcacttgatgcatgtagcttgctcatggatacatcagatggtgaaaaattctctaaatcttgtcaagaacatttccttgctaagagtggccactcacctatgaatgtagtacaaggattggatttcactcggacggcttctattttttcttggcttgaaagtgggtttatgaaggcatatgatcgtgcagagaagatatcacatgtattacggaaaagtgatgataacaccgagatgccagatgcagtggatgttacattccaaactgctttgtaa

SEQ ID NO: 2 - protein, 538 aa, Zea mays
MDEEEREEVGEYKLQGLVGRGSFAEVHRAAHRRTGAPAAVKAIDRRLVDKRVHDGILQEREILRSIDHPNILRLLDTIDTTNMMYLVLEYCDGGDLDAFLHKHGRLPEAVAKDLMRQLAEGLKVLRGRNIVHRDLKPQNLLLSTNGDDIVLKIGDFGFARSLVHENLAATICGSPYYMAPEIWQGKDYDAKSDLWSVGIILFQLVTGKLPFTGSNAFQLHQNVMATDDLNFPSEIEADLCPDCIDLCRRLLHRDPKKRISFEEIFNHKFLATTRKCYESCHAVELRDNCQTITSPVVLKTKSGSIESKNPKVFDSWEWIEREYVLVPENCTSMEMSSLNIKSTKDDMDTRTVSYGRSTGKGSVQNQIRDFTDTATGVQSHGCAPVVISQESATVDCRRGKPPDYITRLHSLNQYVLVLADLAREKLSKGLYLEALSIELVLLTIWKEALDACSLLMDTSDGEKFSKSCQEHFLAKSGHSPMNVVQGLDFTRTASIFSWLESGFMKAYDRAEKISHVLRKSDDNTEMPDAVDVTFQTAL

SEQ ID NO: 3 - DNA, 1803 bp, Zea mays
  source 1..1803: mol_type = other DNA
atgatgtccttccgttcgaaggacgcacatgcacaaacaagacttaactggccatggagaagccaatcgccgttatcagcgcagctgcttgttgatattccacctgaaatagagttgtccgactaccggagattgccaagttctcgaagcgagagcccatctggacttctccatgacgaaggcgtcaaggaagaacctatccctgatctggacattttctttgaaaggctttacgaatatttctgcgcaaaagggctcaggtgtatcattaccaaatggataatagagatcctcaatgtactttttatggtatgctgtatcgggttctttttcttatttgttgattgggatactcttattcatttgaaatgcggtgtggaggcacttgagtctggggagaaaccatgtgatcttatgaaggccattaagcatgatccattagttcccttcacattgcccaaaatgataactgttggatcaatggtcataatgacagcttatggacttaccaactttctcaagttctttgtacagttaagaagtacactcaatgttcgtcagttctactatgacagacttaaggtcaatgatcttgagattcaaactatatcatggcccaaaataattgagaaagttgtcctgctccagaaatctcaaaaactatgtgttgttagggatctctcagagcatgatattatcatgagaatcatgaggaaagaaaattatttgattgggatggtcaataaaggcattctttcttttccaattcattcttgtgtgcctggggctggcccaactggatcacatgagcatggcaggagaaactatctgatacttccaaagtcccttgaatggaccttaaactggtgcatctttcaaagtatgttcgatagtaaattttgtgtaaggaaagagtttttgacaagcccagatgtgttgagaaagcgacttatatttgttggcattgcaatgcttatcctatcaccctgccttgtgatatttccattggtgtacgtcattctaaggcatgctgaagaaatttataatcaccctagtacagcatcatctagacggtggtcaaacttatcaaggtggatctttagggaatataacgaggttgatcacttctttagacacagaatgaacaacagcgctgttcattctttaaattacttgaagcaatttccaaccccactggtttccatcatggctaaatttgtatcttttgtttctggtggcttggctggtgctctaataatcattggatttgtgggtgaatctattcttgaaggacatatttttggaagaaatcttctctggtatactattgtttttggaacaatagcagctataagtcgaaaagtagtggccgatgagcttcaagtttttgacccagaaggggccatgtgcctcgctgttcaccaaacacattacatgccaaagcgatggcgtggtaaagagaacagcgaacttgtacgaagagaatttgagacattatttccgtatactataatcatgctacttgaagagatggcctccattttcatcactccttacttgcttatttctgaggtacctaagcgagttgatgacattctgcgcttcatctcagacttcacaatatatgtagacggagtgggagatgtatgcagtctaagcttgtttaacttcaaaaaacatggaaatagaaactatgggtcaccattcaatgctcttaaaggactcaggagctcccaagggaagatggagaaatcattcttaaggtacatgcagtttcaagtatttcttttacttttttactag

SEQ ID NO: 4 - protein, 600 aa, Zea mays
MMSFRSKDAHAQTRLNWPWRSQSPLSAQLLVDIPPEIELSDYRRLPSSRSESPSGLLHDEGVKEEPIPDLDIFFERLYEYFCAKGLRCIITKWIIEILNVLFMVCCIGFFFLFVDWDTLIHLKCGVEALESGEKPCDLMKAIKHDPLVPFTLPKMITVGSMVIMTAYGLTNFLKFFVQLRSTLNVRQFYYDRLKVNDLEIQTISWPKIIEKVVLLQKSQKLCVVRDLSEHDIIMRIMRKENYLIGMVNKGILSFPIHSCVPGAGPTGSHEHGRRNYLILPKSLEWTLNWCIFQSMFDSKFCVRKEFLTSPDVLRKRLIFVGIAMLILSPCLVIFPLVYVILRHAEEIYNHPSTASSRRWSNLSRWIFREYNEVDHFFRHRMNNSAVHSLNYLKQFPTPLVSIMAKFVSFVSGGLAGALIIIGFVGESILEGHIFGRNLLWYTIVFGTIAAISRKVVADELQVFDPEGAMCLAVHQTHYMPKRWRGKENSELVRREFETLFPYTIIMLLEEMASIFITPYLLISEVPKRVDDILRFISDFTIYVDGVGDVCSLSLFNFKKHGNRNYGSPFNALKGLRSSQGKMEKSFLRYMQFQVFLLLFY

SEQ ID NO: 5 - DNA, 276 bp, Zea mays
  source 1..276: mol_type = other DNA
atggccgcggaggcagatcagaaagtcgtggtgcacgtgcggtcaacgggcgatgcgccgatcctgaagcaatccaaattcaagatttcaggacgagataagtttctgaaggtcatagagtttcttcgtcgacaactgcatcaggatacactgtttgtctatatcaacagtgcattttcgccaaacccagatgaactggtaatagacttgtataataactttggaattgatgggaagctagtggtaaattatgctttatcggcagcatggggctaa

SEQ ID NO: 6 - protein, 91 aa, Zea mays
MAAEADQKVVVHVRSTGDAPILKQSKFKISGRDKFLKVIEFLRRQLHQDTLFVYINSAFSPNPDELVIDLYNNFGIDGKLVVNYALSAAWG

SEQ ID NO: 7 - DNA, 360 bp, Zea mays
  source 1..360: mol_type = other DNA
atggccaggacctctttcaaaatggagcacgacctcgagaaaaggcagtccgaatctgccaggatcagggataagtatcccgacagaattcctgtgattgttgagaaagctggcaaaactgatgtccccgagattgataagaagaagtaccttgtccctgctgatctcactgtcggccagtttgtctatgtggtgaggaagagaatcaagctgagcccagagaaggccatctttgtctttgtgaagaacaccttgccaccaactgcctctttgatgtctgcaatctacgaagagaacaaggacgaggatggcttcctttacatgacctacagtggtgaaaacaccttcggctctgcctag

SEQ ID NO: 8 - protein, 119 aa, Zea mays
MARTSFKMEHDLEKRQSESARIRDKYPDRIPVIVEKAGKTDVPEIDKKKYLVPADLTVGQFVYVVRKRIKLSPEKAIFVFVKNTLPPTASLMSAIYEENKDEDGFLYMTYSGENTFGSA

SEQ ID NO: 9 - DNA, 1557 bp, Zea mays
  source 1..1557: mol_type = other DNA
atggcgcaaggtgaggagagctcgtggtgcatggcgagcagcgatcatggtagagctgcaccattcgtccaagcagtcgcgtgtggaacccaaaggcgcgcttctgccgcacaacctgccaatttcttggagttgcaacctgcagcagctgctccttattactttggtgagttggaggaggcccttatccatggcacccgtacatctgttgatcacggcatgatcggaagtgatgtggacacagagcgtaagacagctggataccttgcagctaggccccccacactggagatcttcccttcatggccaatgagtcatctacagcagccatacagtggtaactcgcactcagtagggagcaccactgagtcgagctcaggtcagaacagtatgccacagaccgagctggtgtccccagggagcatgagggccgactctggacagcgtcaggagcttttgatggtgactgttgatgattacaactacgagcaaggactcggagccgcagcaaccacagctccaatctttcagcagcatgcagcaggccaagacaagggaaagcatggatccacaaggaaagacggcaggctgttagacgccaagaccgagaggcgattggcccagaacagagaagctgcgaggaagagcaggctgagaaaaaaggcctacgtacagcagctagagacgagtcggatcaggcttcagcaaatcgagcaggagctgcatggagcacgctcacagggtctgttcccgggaggaggtggtgcggctggagacttgagttcaggcgctgtgattttcgacatggagtacgcccggtggctagaagacgacaccaagcacatgacggagctccaggccgtgctgcagccccagatcatcgacgcgaacctgggggccatcgtcgaggactgcatgcggcactacgacgagctgttccacctgagggccatgctcgcaagatccgacgtgttccacctgatgaccggcctgtgggcgaccacggccgagcgatgcttcctctggatgggagggttccgtccttctgaaatcctcaagatgctgataccgcagctcgaccctctcgcggagccgcagctgatagggatgtacaatctgcaacggtcgtcggagcagaccgaggaggcgctcgtgcaggggcttcagcagctgcaccagtcgctggcggacgccgtgggcgctagtcctctcagcgacggcgcgaatgtcgccaactacaccgctctcatggccctggcacttgacaggctcgacaccctcgagagcttttaccgccaggcggacagcctgaggcagcagaccttgcaccagatgcggcggatcctgacgacccgccagacggctcggtgcttcgtctccatcagcgagtaccaccgccgcctccgcgccctcagctccgtctgggcatcgtcccgccctccaactcctccaagcgagggcgtcgttgcggccgcagagaatgttagccctacagggacgacgaccgagcaagctctccctccgtaccaccatagccagccgttccctggcttctga

SEQ ID NO: 10 - protein, 518 aa, Zea mays
MAQGEESSWCMASSDHGRAAPFVQAVACGTQRRASAAQPANFLELQPAAAAPYYFGELEEALIHGTRTSVDHGMIGSDVDTERKTAGYLAARPPTLEIFPSWPMSHLQQPYSGNSHSVGSTTESSSGQNSMPQTELVSPGSMRADSGQRQELLMVTVDDYNYEQGLGAAATTAPIFQQHAAGQDKGKHGSTRKDGRLLDAKTERRLAQNREAARKSRLRKKAYVQQLETSRIRLQQIEQELHGARSQGLFPGGGGAAGDLSSGAVIFDMEYARWLEDDTKHMTELQAVLQPQIIDANLGAIVEDCMRHYDELFHLRAMLARSDVFHLMTGLWATTAERCFLWMGGFRPSEILKMLIPQLDPLAEPQLIGMYNLQRSSEQTEEALVQGLQQLHQSLADAVGASPLSDGANVANYTALMALALDRLDTLESFYRQADSLRQQTLHQMRRILTTRQTARCFVSISEYHRRLRALSSVWASSRPPTPPSEGVVAAAENVSPTGTTTEQALPPYHHSQPFPGF